(19) United States
(12) Patent Application Publication
(10) Pub. No.: US 2016/0245789 A1
(43) Pub. Date: Aug. 25, 2016

(54) NANO-GAP ELECTRODE AND METHODS FOR MANUFACTURING SAME

(71) Applicant: Quantum Biosystems Inc., Tokyo (JP)

(72) Inventors: Shuji Ikeda, Tokyo (JP); Mark Oldham, Emerald Hills, CA (US); Eric Nordman, Palo Alto, CA (US)

(21) Appl. No.: 15/048,810

(22) Filed: Feb. 19, 2016

Related U.S. Application Data
(63) Continuation of application No. PCT/IB2014/002143, filed on Aug. 26, 2014.

(30) Foreign Application Priority Data
Aug. 27, 2013 (JP) 2013-176132
Aug. 28, 2013 (JP) 2013-177051

(51) Int. Cl. G01N 33/487; C23C 16/34; C12Q 1/68; C23C 16/56; C23C 14/06; C23C 14/58; G01N 27/447; C23C 16/06

(52) U.S. Cl. CPC G01N 33/48721; G01N 27/44704; G01N 27/44791; C23C 16/345; C23C 16/06; C23C 16/56; C23C 14/0641; C23C 14/5886; C12Q 1/6869

(57) ABSTRACT

The present disclosure provides methods for forming a nano-gap electrode. In some cases, a nano-gap having a width adjusted by a film thickness of a sidewall may be formed between a first electrode-forming part and a second electrode-forming part using sidewall which has contact with first electrode-forming part as a mask. Surfaces of the first electrode-forming part, the sidewall and the second electrode-forming part may then be exposed. The sidewall may then be removed to form a nano-gap between the first electrode-forming part and the second electrode-forming part.

### CROSS-REFERENCE

This application is a Continuation Application of International Patent Application No. PCT/IB2014/002143, filed Aug. 26, 2014, which claims priority to Japanese Patent Application Nos. JP 2013-176132, filed Aug. 27, 2013, and JP 2013-177051, filed Aug. 28, 2013, each of which is entirely incorporated herein by reference.

### BACKGROUND

In recent years, an electrode structure (hereinafter referred to as a nano-gap electrode) in which a nanoscale gap is formed between opposed electrodes has been a focus of attention. Accordingly, active research is being conducted on electronic devices, biodevices, and the like using nano-gap electrodes. For example, an analytical apparatus for analyzing the nucleotide sequence of DNA utilizing a nano-gap electrode has been conceived in the field of biodevices (see, for example, WO2011/108540).

In this analytical apparatus, single-stranded DNA is passed through a nanoscale (hollow) gap (hereinafter referred to as a nano-gap) between electrodes of a nano-gap electrode. Current flowing through the electrodes may be measured when bases of the single-stranded DNA pass through the nano-gap between the electrodes, thereby enabling the bases constituting the single-stranded DNA to be determined on the basis of the current values.

In such an analytical apparatus as mentioned above, the detectable value of a current decreases if the distance between the electrodes of the nano-gap electrode increases. This makes it difficult to analyze samples with high sensitivity. Accordingly, it is desired that the nano-gap between the electrodes be formed to a small size.

Existing methods for manufacturing a nano-gap electrode include a method in which a metal mask, such as a titanium mask, formed on an electrode forming layer made from gold or the like, is patterned by irradiating the mask with a focused ion beam; the underlying electrode layer exposed through this patterned metal mask may be dry-etched, and a nano-gap may be formed from the electrode layer, thereby forming a nano-gap electrode (see, for example, Japanese Patent Laid-Open No. 2004-247203).

In such a method for manufacturing a nano-gap electrode as described above, the exposed electrode layer not covered with the patterned metal mask is dry-etched to form a gap to serve as the nano-gap in the electrode layer. Hence the minimum width of the gap (mask width gap) formed in the electrode layer is the smallest width wherein the metal mask can be patterned. The method therefore has a problem in that it is difficult to form a nano-gap (a conventional nano-gap) smaller than that width using standard lithographic methods. Accordingly, in recent years, there has been a desire for the development of a new manufacturing method capable of forming not only a nano-gap of the same width as a conventional nano-gap, but also a nano-gap even smaller than a conventional nano-gap.

Hence, an object of the present invention is to describe a method for manufacturing a nano-gap electrode capable of forming not only a nano-gap of the same width as a conventional nano-gap, but also a nano-gap that is even smaller in width than a conventional nano-gap.

The present invention relates to a nano-gap electrode and to a method of manufacturing the nano-gap electrode.

Focused ion beam, e-beam and nano-imprint technologies have been described as being useful for creating nanochannels which may have widths and depths of 20 nanometers (nm), potentially being at least 10 nm. Systems have been described wherein the channel width is less than the radius of gyration for double stranded DNA; but systems and methods with width sufficiently small as to be less than the radius of gyration of single stranded DNA have not been described.

A need exists for nanochannels with dimensions sufficiently small as to allow access by sample biomolecules to nanogap structures, allowing interrogation of a higher percentage of biomolecules, while also potentially preventing secondary structure from forming betwixt different parts of the biomolecule.

In such a method for manufacturing a nano-gap electrode as described above, however, the exposed electrode layer not covered with the patterned metal mask may be dry-etched to form a gap to serve as the nano-gap in the electrode layer. Hence, the minimum width of the gap (which corresponds to the width of the mask gap) formed in the electrode layer is the minimum width for which the metal mask can be patterned. This method therefore has a problem in that it is difficult to form a nano-gap smaller than the width of the smallest feature which may be formed on the metal mask.

### SUMMARY

The present disclosure provides devices, systems and methods for nano-gap electrodes and nanochannel systems. Methods provided herein may be used to form a nano-gap electrode having a nano-gap that is smaller than a gap formed using other methods currently available.

In some embodiments, a method of manufacturing a nano-gap electrode includes using a sidewall disposed on an electrode-forming part as a mask, and forming a nano-gap having a width adjusted by a film thickness of the sidewall on the electrode-forming part.

In other embodiments, a method of manufacturing a nano-gap electrode includes forming a sidewall on a lateral wall of a first electrode-forming part formed on a substrate, and then forming a second electrode-forming part so as to abut on the sidewall, thereby disposing the sidewall between the first electrode-forming part and the second electrode-forming part; and exposing surfaces of the first electrode-forming part, the sidewall and the second electrode-forming part and removing the sidewall, thereby forming a nano-gap between the first electrode-forming part and the second electrode-forming part.

In additional embodiments a method of manufacturing a nano-gap electrode includes disposing a gap-forming mask having lateral walls opposed to each other across a gap on an electrode-forming part; forming sidewalls on both of the lateral walls of the gap-forming mask, and exposing the electrode-forming part between the sidewalls; and removing the electrode-forming part exposed between the sidewalls to form a nano-gap therebetween.

In further embodiments a method of manufacturing a nano-gap electrode includes removing sidewalls provided in a gap-forming mask to form a gap in the gap-forming mask to expose an electrode-forming part out of the gap; and removing the electrode-forming part exposed out of the gap to form a nano-gap within the gap.

In other embodiments a method of manufacturing a nano-gap electrode includes forming a sidewall on a lateral wall of a sidewall-forming mask disposed on an electrode-forming part, and then removing the sidewall-forming mask to vertically build the sidewall; forming a gap-forming mask so as to surround the sidewall; removing the sidewall to form a gap in the gap-forming mask, and exposing the electrode-forming part out of the gap; and removing the electrode-forming part exposed out of the gap to form a nano-gap within the gap.

In additional embodiments, a method of manufacturing a nano-gap electrode includes forming a sidewall on a lateral wall of a first gap-forming mask disposed on an electrode-forming part, and then forming a second gap-forming mask so as to abut on the sidewall, thereby disposing the sidewall between the first gap-forming mask and the second gap-forming mask; exposing surfaces of the first gap-forming mask, the sidewall and the second gap-forming mask and removing the sidewall, thereby forming a gap between the first gap-forming mask and the second gap-forming mask; and removing the electrode-forming part within the gap to form a nano-gap within the gap.

According to the present invention, it is possible to form a nano-gap having a width adjusted by the film thickness of a sidewall. Consequently, it is possible to form not only a nano-gap that is the same width as a conventional nano-gap, but also a nano-gap that is even smaller in width than a conventional nano-gap.

According to an aspect of the present invention, a method of manufacturing a nano-gap electrode may include: film-forming a compound-generating layer on opposing electrode-forming parts, and then performing a heat treatment; reacting the electrode-forming parts with a compound-generating layer; forming two volumetrically expanded opposed electrodes by the reaction; and bringing sidewalls of the electrodes closer to each other by volumetric expansion, thereby forming a nano-gap between the electrodes.

According to another aspect of the present disclosure, a method of manufacturing a nano-gap electrode includes:

forming a mask selected in conformity with a specific width on a pair of opposing electrode-forming parts located on a substrate;

forming a film of a compound-generating layer on the electrode-forming parts;

performing a heat treatment to react the compound-generating layer with the electrode-forming parts to form two electrodes opposed to each other and penetrating underneath the mask by volumetric expansion resulting from the reaction, thereby bringing sidewalls of the electrodes closer to each other than the width of the mask, by the volumetric expansion; and

removing the mask and any unreacted portions of the electrode-forming parts remaining in the region previously underneath the mask, thereby forming a nano-gap between the electrodes.

According to another aspect of the present invention, a method of manufacturing a nano-gap electrode includes:

forming two electrode-forming parts disposed opposing each other across a gap on a substrate;

forming a film of a compound-generating layer on the electrode-forming parts; and

performing a heat treatment to cause a reaction to the compound-generating layer with the electrode-forming parts to form two electrodes volumetrically expanded by the reaction and opposed to each other, thereby bringing sidewalls of the electrode parts closer to each other by volumetric expansion to form a nano-gap smaller than the gap.

In another embodiment, a gap between electrodes may be made smaller by as much as the amount of volumetric expansion of the electrodes. Consequently, it is possible to provide a nano-gap electrode having a nano-gap that is even smaller than a gap formed by standard lithographic processing, and to provide a method for manufacturing a nano-gap electrode.

In some embodiments, methods such as those described herein as being useful for the formation of a nanogap electrode structure may be utilized to form a nano channel which may be smaller than may be formed using conventional semiconductor processes, such as e-beam, ion beam milling, or nanoimprint lithography.

An aspect of the present disclosure provides a method for manufacturing a sensor having at least one nano-gap, comprising (a) providing a first electrode-forming part adjacent to a substrate, a sidewall adjacent to the first electrode-forming part, and a second electrode-forming part adjacent to the sidewall; (b) removing the sidewall, thereby forming a nano-gap between the first electrode-forming part and the second electrode-forming part; and (c) preparing the first electrode-forming part and the second electrode-forming part for use as electrodes that detect a current across the nano-gap when a target species is disposed therebetween. In an embodiment, the current is a tunneling current.

In an embodiment, preparing the first electrode-forming part and the second electrode-forming part for use as the electrodes comprises removing at least a portion of the first electrode-forming part and the second electrode-forming part to provide the electrodes. In another embodiment, the first and/or second electrode-forming part is formed of a metal nitride. In another embodiment, the first and/or second electrode-forming part is formed of titanium nitride. In another embodiment, the substrate comprises a semiconductor oxide layer adjacent to a semiconductor layer. In another embodiment, the semiconductor is silicon.

In an embodiment, the sidewall has a width that is less than or equal to about 2 nanometers. In another embodiment, the width is less than or equal to about 1 nanometer. In another embodiment, the width is greater than about 0.5 nanometers.

In an embodiment, the method further comprises, prior to (c), exposing surfaces of the first electrode-forming part, the sidewall and the second electrode-forming part.

In an embodiment, the method further comprises, prior to (b), removing a portion of the sidewall such that a cross section of the sidewall between first electrode-forming part and the second electrode-forming part has a quadrilateral shape.

In an embodiment, the method further comprises forming a channel intersecting the nano-gap. In another embodiment, the channel is a covered channel.

Another aspect of the present disclosure provides a method for forming a sensor having at least one nano-gap, comprising (a) disposing a gap-forming mask having lateral walls opposed to each other across a gap on an electrode-forming part that is adjacent to a substrate, wherein the gap has a first width; (b) forming sidewalls on the lateral walls of the gap-forming mask, wherein the electrode-forming part is exposed between the sidewalls; (c) removing a portion of the electrode-forming part exposed between the sidewalls to form a nano-gap therebetween, wherein the nano-gap has a second width that is less than the first width; (d) removing the sidewalls to expose portions of the electrode-forming part separated by the nano-gap; and (e) preparing the portions of the electrode-forming part for use as electrodes that detect a current across the nano-gap when a target species is disposed therebetween. In an embodiment, the current is a tunneling current.

In an embodiment, preparing the portions of the electrode-forming part for use as the electrodes comprises removing the portions of the electrode-forming part to provide the electrodes. In another embodiment, the substrate comprises a semiconductor oxide layer adjacent to a semiconductor layer. In another embodiment, the semiconductor is silicon.

In an embodiment, the second width is less than or equal to about 2 nanometers. In another embodiment, the second width is less than or equal to about 1 nanometer. In another embodiment, the second width is greater than about 0.5 nanometers.

In an embodiment, the target species is a nucleic acid molecule, and wherein the second width is less than a diameter of the nucleic acid molecule. In another embodiment, the gap-forming mask and the sidewalls are formed of different materials.

In an embodiment, the method further comprises forming a channel intersecting the nano-gap. In another embodiment, the channel is a covered channel.

Another aspect of the present disclosure provides a method for forming a sensor having at least one nano-gap, comprising (a) providing a mask comprising a sidewall, wherein the sidewall is disposed adjacent to an electrode-forming part that is adjacent to a substrate; (b) removing the sidewall to form a gap in the mask, wherein the gap exposes a portion of the electrode-forming part; (c) removing the portion of the electrode-forming part to form a nano-gap; (d) removing the mask to expose portions of the electrode-forming part separated by the nano-gap; and (e) preparing the portions of the electrode-forming part for use as electrodes that detect a current across the nano-gap when a target species is disposed therebetween. In an embodiment, the current is a tunneling current. In another embodiment, the target species is a nucleic acid molecule, and wherein the sidewall has a width that is less than a diameter of the nucleic acid molecule.

In an embodiment, preparing the portions of the electrode-forming part for use as the electrodes comprises removing the portions of the electrode-forming part to provide the electrodes.

In an embodiment, (a) comprises (i) providing the sidewall on a lateral wall of a first mask disposed adjacent to the electrode-forming part, (ii) removing the first mask, and (iii) forming a second mask adjacent to the sidewall, wherein the mask comprises at least a portion of the second mask. In another embodiment, removing the first mask exposes the electrode-forming part. In another embodiment, the second mask covers the sidewall. In another embodiment, subsequent to removing the first mask, the sidewall is a free-standing sidewall having a width that is less than or equal to about 10 nanometers (nm), 5 nm, 4 nm, 3 nm, 2 nm, 1 nm, 0.9 nm, 0.8 nm, 0.7 nm, 0.6 nm or 0.5 nm.

In an embodiment, (a) comprises (i) providing the sidewall on a lateral wall of a first mask disposed adjacent to the electrode-forming part, (ii) forming a second mask adjacent to the sidewall, and (iii) etching the second mask, wherein the mask comprises at least a portion of the first mask and the second mask. In another embodiment, forming the second mask adjacent to the sidewall includes the second mask covering the first mask and the sidewall. In another embodiment, etching the second mask comprises etching the first mask and/or the sidewall.

In an embodiment, the method further comprises forming a channel intersecting the nano-gap. In another embodiment, the channel is a covered channel.

In an embodiment, the substrate comprises a semiconductor oxide layer adjacent to a semiconductor layer. In another embodiment, the semiconductor is silicon.

In an embodiment, (a) further comprises providing a side-wall forming layer and etching the side-wall forming layer to form the sidewall.

In an embodiment, the nano-gap has a width that is less than or equal to about 2 nanometers. In another embodiment, the width is less than or equal to about 1 nanometer. In another embodiment, the width is greater than about 0.5 nanometers.

In an embodiment, the method further comprises forming a channel intersecting the nano-gap. In another embodiment, the channel is a covered channel.

Another aspect of the present disclosure provides a method of manufacturing a nano-gap electrode sensor, comprising (a) providing a film having a first material on an electrode-forming part having a second material, wherein the electrode-forming part is disposed adjacent to a substrate; (b) heating the film to react the first and second materials, thereby forming two electrode parts volumetrically expanded and opposed to each other, wherein each of the electrode parts has a sidewall; (c) bringing sidewalls of the electrode parts towards each other by volumetric expansion, thereby forming a nano-gap between the electrode parts; and (d) preparing the electrode parts for use as electrodes that detect a current across the nano-gap when a target species is disposed therebetween. In an embodiment, the current is a tunneling current.

In an embodiment, preparing the electrode parts for use as the electrodes comprises removing at least a portion of the electrode parts to provide the electrodes. In another embodiment, (a) comprises (i) forming a mask selected in conformity with a width of the electrode-forming part, (ii) forming the film on the electrode-forming part. In another embodiment, upon forming two electrode parts, the two electrode parts penetrate into the mask by volumetric expansion resulting from the reaction, thereby bringing sidewalls of the electrode parts towards each other. In another embodiment, the method further comprises removing the mask and unreacted portion(s) of the electrode parts remaining in a lower region of the mask, thereby forming a nano-gap between the electrode parts.

In an embodiment, the method further comprises forming a channel intersecting the nano-gap. In another embodiment, the channel is a covered channel.

Another aspect of the present disclosure provides a method of manufacturing a sensor having at least one nano-gap electrode, comprising (a) providing two electrode-forming parts adjacent to a substrate, wherein the electrode-forming parts are disposed opposite one another across a gap having a first width; (b) forming a film of a compound-generating layer on the electrode-forming parts; (c) performing a heat treatment to facilitate a reaction between the compound-generating layer and at least one of the electrode-forming parts to form at least one electrode part volumetrically expanded by the reaction, thereby bringing sidewalls of the electrode-forming parts towards each other by volumetric expansion to form a nano-gap having a second width smaller than the first width; and (d) preparing the electrode-forming parts for use as electrodes that detect a current across the nano-gap when a target species is disposed therebetween. In an embodiment, the current is a tunneling current.

In an embodiment, preparing the electrode-forming parts for use as the electrodes comprises removing the portions of the electrode-forming part to provide the electrodes. In another embodiment, the compound-generating layer is a silicide-generating layer, wherein (c) comprises a silicidation of the electrode-forming parts during the reaction, and wherein the electrode-forming parts expand volumetrically during the silicidation.

In an embodiment, the second width is less than or equal to about 2 nanometers. In another embodiment, the second width is less than or equal to about 1 nanometer. In another embodiment, the second width is greater than about 0.5 nanometers.

In an embodiment, the target species is a nucleic acid molecule, and wherein the second width is less than a diameter of the nucleic acid molecule.

In an embodiment, (c) comprises the reaction between the compound-generating layer and both of the electrode-forming parts. In another embodiment, (c) comprises the reaction between the compound-generating layer and only one of the electrode-forming parts.

In an embodiment, the method further comprises forming a channel intersecting the nano-gap. In another embodiment, the channel is a covered channel.

Another aspect of the present disclosure provides a nano-gap electrode sensor comprising at least two electrode parts disposed oppositely across a nano-gap on a substrate, wherein opposed sidewalls of the electrode parts gradually come closer to each other and a width between the sidewalls narrows gradually, and wherein the electrodes are adapted to detect a current across the nano-gap when a target species is disposed therebetween. In an embodiment, the current is a tunneling current.

In an embodiment, the electrode parts are formed of a metal silicide. In another embodiment, the nano-gap is formed into a trailing curved shape in which the distance between the sidewalls of the electrode parts widens gradually as the nano-gap approaches the substrate. In another embodiment, the sidewalls include outwardly expanding portions in contact with the substrate.

In an embodiment, the sensor further comprises a channel intersecting and in fluid communication with the nano-gap. In another embodiment, the channel is a covered channel.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is a schematic view illustrating the configuration of a nano-gap electrode manufactured by a manufacturing method;

FIGS. 2A-2F are schematic views used for description of a method for manufacturing the nano-gap electrode of FIG. 1;

FIGS. 3A-3F are schematic views used for description of a method for manufacturing a nano-gap electrode of FIG. 1;

FIG. 4 is a schematic view illustrating the configuration of a nano-gap electrode manufactured by a manufacturing method;

FIG. 5 is a schematic view used for description of a method for manufacturing a nano-gap electrode of FIG. 4;

FIGS. 6A-6C are schematic views used for description of a method for manufacturing a nano-gap electrode according of FIG. 4;

FIGS. 7A-7C are schematic views used for description of a method for manufacturing a nano-gap electrode of FIG. 4;

FIGS. 8A-8C are schematic views used for description of a method for manufacturing a nano-gap electrode;

FIGS. 9A-9B are schematic views used for description of a method for manufacturing a nano-gap electrode of FIG. 8;

FIGS. 10A-10C are schematic views used for description of a method for manufacturing a nano-gap electrode;

FIGS. 11A-11B are schematic views used for description of a method for manufacturing a nano-gap electrode of FIG. 10;

FIGS. 12A-12D are schematic views used for description of a method for manufacturing a nanogap of FIG. 1;

FIGS. 13A-13F are additional schematic views for describing the method associated with FIGS. 12A-12C;

FIG. 14 is a schematic view showing a nano-gap electrode;

FIG. 15 is a schematic view showing a configuration in which an electrode-forming part and a mask are formed on a substrate;

FIGS. 16A-16F is a schematic view used for describing a method for manufacturing a nano-gap electrode;

FIGS. 17A-17F is another schematic view used for describing a method for manufacturing a nano-gap electrode;

FIG. 18 is a schematic view showing the configuration of a nano-gap electrode according to another embodiment;

FIGS. 19A-19D is a schematic view used to describe a method for manufacturing the nano-gap electrode;

FIGS. 20A-20C is another schematic view used for describing a method for manufacturing a nano-gap electrode;

FIGS. 21A-21C is a schematic top view representation showing some alternative electrode shapes;

FIGS. 22A-22F is a schematic representation of cross sections used for describing a method for manufacturing a nano-gap electrode with an integrated channel for delivering the DNA to the nano-gap electrode;

FIG. 23 is a schematic top view showing a configuration for an integrated channel for delivering DNA to one or more nano-gap electrodes;

FIGS. 24A-24C is a schematic view used to describe a method for manufacturing the nano-gap electrode using a single side expansion approach; and

FIGS. 25A-25C is a schematic view used to describe a method for manufacturing the nano-gap electrode using a vertical electrode orientation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap.”

The term “electrode-forming part,” as used herein, generally refers to a part or member that may be used to generate an electrode. The electrode-forming part may be the electrode or may be part of the electrode. For example, the electrode-forming part is a first electrical conductor that is in electrical communication with a second electrical conductor. In another example, the electrode-forming part is an electrode.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded.

The present disclosure provides methods for forming sensors with nano-gap electrodes, which may be used in various applications, such as detecting a biomolecule (e.g., nucleic acid molecule). Nano-gap electrodes formed according to methods provided herein may be used to sequence a nucleic acid molecule, such deoxyribonucleic acid (DNA), ribonucleic acid (RNA), or variants thereof.

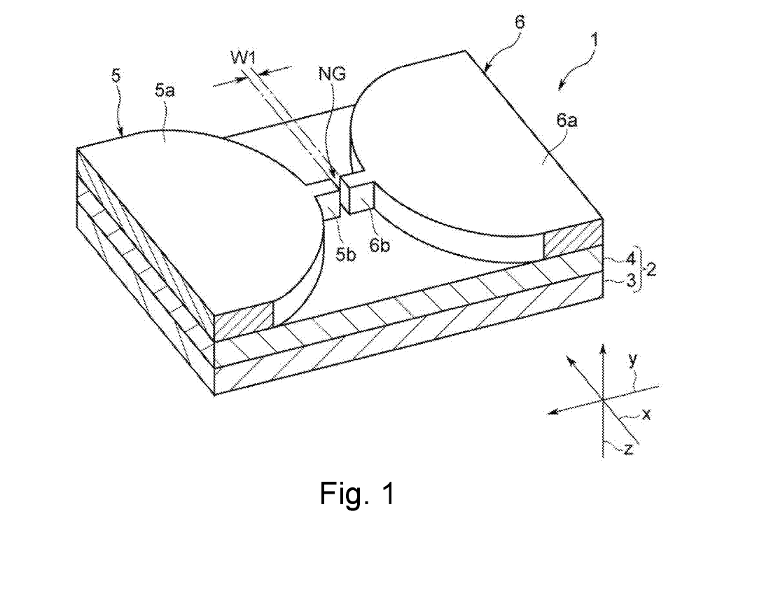

FIG. 1 shows a nano-gap electrode 1 which may be formed according to methods provided herein. In this nano-gap electrode 1, opposed electrodes 5 and 6 are disposed on a substrate 2. A nano-gap NG (or pore) with a width W1 which is of nanoscale (no larger than, for example, 1000 nanometers) is formed between electrodes 5 and 6. Nano-gap electrode 1 when manufactured by the manufacturing methods described herein may allow, for example, a nano-gap NG to be formed with a width W1 of 0.1 nanometers (nm) to 30 nm, or no greater than 2 nm, 1 nm, 0.9 nm, 0.8 nm, 0.7 nm, 0.6 nm, or 0.5 nm or of any other widths as described herein. In some cases, W1 is less than a diameter of a target species, which may be a biomolecule (e.g., DNA or RNA).

Substrate 2 may be composed of, for example, a silicon substrate 3 and a silicon oxide layer 4 formed thereon. As an alternative, substrate 2 may include other semiconductor materials(s), including a Group IV or Group III-V semiconductor, such as germanium or gallium arsenide, including oxides thereof. Substrate 2 can have a configuration in which two electrodes 5 and 6 forming a pair may be formed on silicon oxide layer 4. Electrodes 5 and 6 may comprise a metal material, such as titanium nitride (TiN), and in some embodiments may be formed almost bilaterally symmetrically across nano-gap NG on substrate 2. In some embodiments, electrodes 5 and 6 have substantially the same configuration and may be composed of leading electrode edges 5b and 6b forming nano-gap NG, and base parts 5a and 6a may be integrally formed with the root portions of the leading electrode edges 5b and 6b. Leading electrode edges 5b and 6b may comprise, for example, rectangular solids, the longitudinal directions of which may extend in a y-direction, and may be disposed so that the apical surfaces of the leading electrode edges 5b and 6b face each other; leading edges 5b and 6b may have curves (not shown).

Base parts 5a and 6a may have protrusions at the central apical ends thereof whereby the leading electrode edges 5b and 6b may be formed. A gently curved surface may be formed toward both sides of each base part 5a and 6a with the central apical end thereof at the center. Thus, base parts 5a and 6a may be formed into a curved shape with leading electrode edges 5b and 6b positioned at the vertexes. Note that electrodes 5 and 6 may be configured so that when a solution containing single-stranded DNA, for example, is supplied from an x-direction orthogonal to the y-direction which may be the longitudinal direction of electrodes 5 and 6 and to a z-direction which may be the vertical direction of electrodes 5 and 6 and may intersects at right angles with this y-direction, the solution may be guided along the curved surfaces of base parts 5a and 6a to leading electrode edges 5b and 6b to enable the solution to reliably pass through nano-gap NG.

Note that for a nano-gap electrode 1 configured as described above, current can be supplied from, for example, a power source (not shown) to electrodes 5 and 6, and values of current flowing across electrodes 5 and 6 can be measured with an ammeter (not shown). Accordingly, a nano-gap electrode 1 allows single-stranded DNA to pass through a nano-gap NG between electrodes 5 and 6 from the x-direction; an ammeter to measure values of currents flowing across electrodes 5 and 6 when bases of single-stranded DNA pass through nano-gap NG between electrodes 5 and 6; and the bases constituting single-stranded DNA may be determined on the basis of the correlated current values.

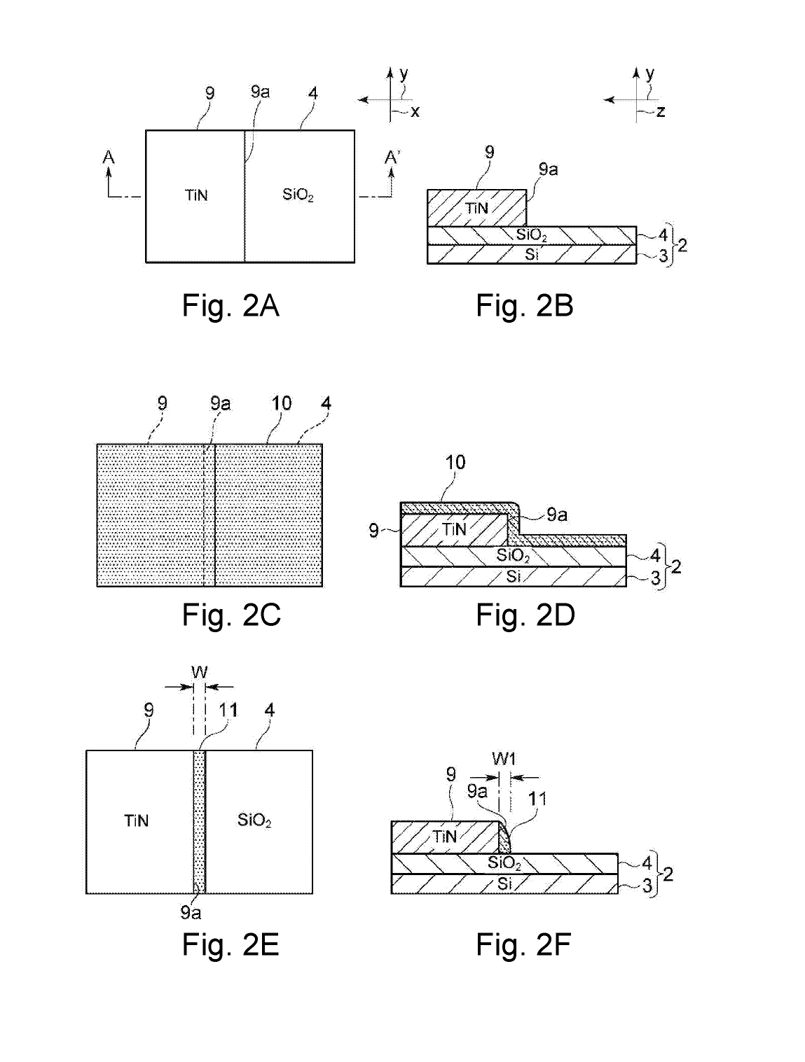

In other embodiments, a method for manufacturing the nano-gap electrode 1 having a nano-gap NG between electrodes 5 and 6 is described herein. Substrate 2 for which the silicon oxide layer 4 may be formed on a silicon substrate 3 may be prepared first, and a quadrilateral first electrode-forming part 9 made from, for example, titanium nitride (TiN) and having a lateral wall 9a may be formed on a predetermined region of silicon oxide layer 4 using a photolithographic technique, as shown in FIG. 2A, and FIG. 2B which shows a lateral cross-sectional view of section A-A′ in FIG. 2A.

Subsequently, as shown in FIG. 2C in which constituent elements corresponding to those of FIG. 2A are denoted by like reference numerals and FIG. 2D in which constituent elements corresponding to those of FIG. 2B are denoted by like reference numerals, a sidewall-forming layer 10 made from a material, such as titanium (Ti) or silicon nitride (SiN), different from the material of the surface (silicon oxide layer 4 in this case) of substrate 2 may be film-formed on first electrode-forming part 9 and exposed portions of substrate 2 by, for example, a CVD (Chemical Vapor Deposition) method. At this time, a sidewall-forming layer 10 may be formed along lateral wall 9a of first electrode-forming part 9. The film thickness of sidewall-forming layer 10 to be formed on the lateral wall 9a may be selected according to a desired width W1 of nano-gap NG. That is, when a nano-gap NG having a small width W1 is formed, sidewall-forming layer 10 may be formed with a small film thickness. On the other hand, when a nano-gap NG having a large width W1 is formed, sidewall-forming layer 10 may be formed with a large film thickness.

Subsequently, sidewall-forming layer 10 film-formed on first electrode-forming part 9 and exposed portions of the substrate 2 may be etched back by, for example, dry etching to leave a portion of sidewall-forming layer 10 along lateral wall 9a of the first electrode-forming part 9. The etching process may be configured to be perpendicular with respect to substrate 2, or may be angled such that a portion of sidewall-forming layer 10 may be at least partially protected from etching by lateral wall 9a of first electrode-forming part 9. Thus, a sidewall 11 may be formed along lateral wall 9a of first electrode-forming part 9, as shown in FIG. 2E in which constituent elements corresponding to those of FIG. 2C are denoted by like reference numerals and FIG. 2F in which constituent elements corresponding to those of FIG. 2D are denoted by like reference numerals. Note that the sidewall 11 formed in this way may thicken gradually from the vertex of lateral wall 9a of first electrode-forming part 9 toward substrate 2. Accordingly, a maximum thickness of sidewall 11 may be of a width W1 corresponding to nano-gap NG to be formed later, as described herein.

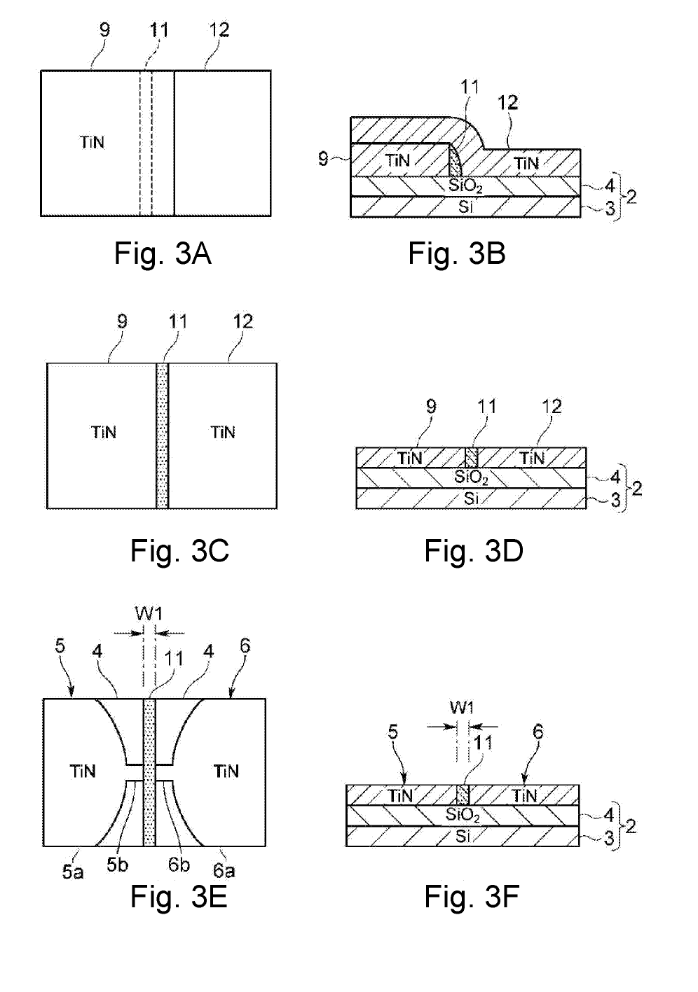

Subsequently, as shown in FIG. 3A in which constituent elements corresponding to those of FIG. 2E are denoted by like reference numerals and FIG. 3B in which constituent elements corresponding to those of FIG. 2F are denoted by like reference numerals, a second electrode-forming part 12 comprising a metal material, such as titanium nitride (TiN), may be formed on first electrode-forming part 9, sidewall 11 and exposed portions of substrate 2 by, for example, a sputtering method. Then, first electrode-forming part 9 and sidewall 11, as well as regions of second electrode-forming part 12 covering first electrode-forming part 9 and sidewall 11, may be polished an may be over polished by planarization processing, such as chemical mechanical polishing or planarization (CMP). Thus, top surfaces of first electrode-forming part 9, sidewall 11 and second electrode-forming part 12 may be exposed, as shown in FIG. 3C in which constituent elements corresponding to those of FIG. 3A are denoted by like reference numerals and FIG. 3D in which constituent elements corresponding to those of FIG. 3B are denoted by like reference numerals.

In some embodiments, the largely inclined upper region of the side surface of sidewall 11 and the parts of second electrode-forming part 12 above sidewall 11 and electrode-forming part 9 may be polished and first electrode-forming part 9, sidewall 11, and second electrode-forming part 12 may be over-polished in the planarization processing until the cross section of sidewall 11 between first electrode-forming part 9 and second electrode-forming part 12 may be formed into a substantially quadrilateral shape. Note that only the regions of second electrode-forming part 12 covering first electrode-forming part 9 and sidewall 11 may be polished, as long as surfaces of all of first electrode-forming part 9, sidewall 11 and second electrode-forming part 12 may be exposed when the planarization processing is performed.

Then, a layer-like resist mask may be formed on the exposed surfaces of first electrode-forming part 9, sidewall 11 and second electrode-forming part 12, and then first electrode-forming part 9 and second electrode-forming part 12 may be patterned using a photolithographic technique. In some cases, the resist mask can include a polymeric material, such as poly(methyl methacrylate) (PMMA), poly(methyl glutarimide) (PMGI), phenol formaldehyde resin, or SU-8 (see Liu et al., “Process research of high aspect ratio microstructure using SU-8 resist,” Microsystem Technologies 2004, V10, (4), 265, which is entirely incorporated herein by reference). The mask may be used to form the gentle curves for base parts 5a and 6a, and protrusions for leading electrode edges 5b and 6b. Thus, electrode 5 having a predetermined shape based in part on first electrode-forming part 9 and electrode 6 having a predetermined shape based in part on second electrode-forming part 12 may be formed, as shown in FIG. 3E in which constituent elements corresponding to those of FIG. 3C are denoted by like reference numerals and FIG. 3F in which constituent elements corresponding to those of FIG. 3D are denoted by like reference numerals, thereby forming a structure in which leading electrode edges 5b and 6b may be disposed opposite to each other across sidewall 11 on substrate 2. The sidewall 11 between leading electrode edges 5b and 6b may be removed by, for example, wet etching. Thus, it is possible to form a nano-gap NG having the same width W1 as the width W1 of sidewall 11 between leading electrode edges 5b and 6b, and manufacture a nano-gap electrode 1 as shown in FIG. 1. Since sidewall 11 may be formed from a material, such as a nitride (N) or, in some cases, a silicon nitride (SiN), different from, for example, silicon oxide layer 4 located on the surface of substrate 2, it is possible to selectively remove only sidewall 11 and reliably leave electrodes 5 and 6 on substrate 2.

In some cases, the first electrode-forming part 9 and the second electrode-forming part 12 are prepared for use as electrodes that detect a current across the nano-gap when a target species (e.g., a biomolecule, such as DNA or RNA) is disposed therebetween. The current can be a tunneling current. Such a current can be detected upon the flow of the target species through the nano-gap. In some cases, a sensing circuit coupled to the electrodes provides an applied voltage across the electrodes to generate a current. As an alternative or in addition to, the electrodes can be used to measure and/or identify the electric conductance associated with the target species (e.g., a base of a nucleic acid molecule). In such a case, the tunneling current can be related to the electric conductance.

In some cases, the sidewall 11 may be formed on lateral wall 9a of first electrode-forming part 9 which may be previously formed on the substrate 2, and second electrode-forming part 12 may be formed on first electrode-forming part 9, sidewall 11 and exposed portions of substrate 2. Thereafter, portions of the second electrode-forming part 12 may be removed so as to expose portions of first electrode-forming part 9 and sidewall 11 covered with second electrode-forming part 12, thereby exposing the first electrode-forming part 9, sidewall 11 and second electrode-forming part 12 on substrate 2. Then, sidewall 11 between first electrode-forming part 9 and second electrode-forming part 12 may be removed to form nano-gap NG therebetween. Thereafter, first electrode-forming part 9 and second electrode-forming part 12 may be patterned to form electrodes 5 and 6 in which the nano-gap NG may be provided between leading electrode edges 5b and 6b.

In such a manufacturing method of the present invention as described above, it is possible to form a nano-gap NG having a desired width W1 by adjusting the film thickness of sidewall 11. In addition, it is possible to form sidewall 11 with an extremely small film thickness. It is therefore possible to form a nano-gap NG having an extremely small width W1 corresponding to width W1 of sidewall 11.

In some embodiments, nano-gap NG having a width W1 may be adjusted by controlling the film thickness of sidewall 11 formed between first electrode-forming part 9 and second electrode-forming part 12 using sidewall 11 disposed adjacent to first electrode-forming part 9 as a mask. Consequently, it is possible to form not only a nano-gap NG with the same width W1 as a conventional nano-gap, but also to form a nano-gap NG that is even smaller in width W1 than a conventional nano-gap.

Note that in the above-described embodiments, second electrode-forming part 12 has been described as being directly formed on the first electrode-forming part 9 in the course of manufacture, as shown in FIG. 3B. In other embodiments, a first electrode-forming part 9 on a surface also comprising a hard mask may be used without directly forming second electrode-forming part 12 on first electrode-forming part 9. Even in this case, it is possible to form second electrode-forming part 12 so as to abut sidewall 11, and dispose sidewall 11 between first electrode-forming part 9 and second electrode-forming part 12. Consequently, it is possible to form nano-gap NG between first electrode-forming part 9 and second electrode-forming part 12 by removing sidewall 11.

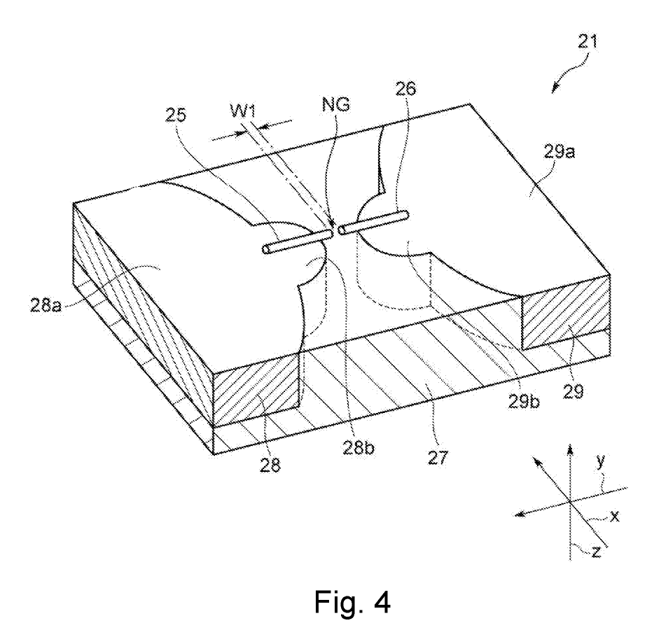

In other embodiments as shown in FIG. 4, which depicts an alternative nano-gap electrode 21, in which columnar electrodes 25 and 26, the apical surfaces of which face each other, are disposed on a substrate 22. A nano-gap NG, the width W1 of which may be nanoscale (no greater than, for example, 1000 nm), may be formed between electrodes 25 and 26. In some embodiments, nano-gap electrode 21 may be manufactured by a manufacturing method as described herein, and nano-gap NG may be formed to a width W1 of 0.1 nm to 30 nm, or no greater than 2 nm, 1 nm, 0.9 nm, 0.8 nm, 0.7 nm, 0.6 nm, or 0.5 nm, or any other width as described herein.

In some embodiments, substrate 22 may comprise a silicon oxide layer 27 formed on, for example, a silicon substrate (not shown), and electrode-supporting parts 28 and 29 may be disposed opposite to each other on silicon oxide layer 27. On a surface of a substrate, one electrode 25 may be disposed on one electrode-supporting part 28, and the another electrode 26 forming a pair with electrode 25 may be disposed on electrode-supporting part 29.

Note that both the electrode-supporting parts 28 and 29 may be made from a material comprising a metal, such as titanium nitride (TiN), and may be formed almost bilaterally symmetrically across a predetermined gap formed above a substrate between electrode supporting parts 28 and 29, wherein the front surfaces of electrode-supporting parts 28 and 29 may be flush with the front surface of silicon oxide layer 27. In some embodiments, electrode-supporting parts 28 and 29 may have substantially the same configuration and may comprise of expanded electrode-supporting parts 28b and 29b whereupon electrodes 25 and 26 may be fixed, and base parts 28a and 29a may be integrally formed in the root portions of the expanded electrode-supporting parts 28b and 29b, wherein expanded electrode-supporting parts 28b and 28b protrude from electrode-forming base parts 28a and 29a. In some embodiments, expanded electrode-forming parts 28b and 29b of electrode-supporting parts 28 and 29 may be formed into a substantially semicircular shape, and electrode-forming base parts 28a and 29a may gently incline toward both lateral portions thereof with the central leading edges of expanded electrode-forming parts 28b and 29b wherein expanded electrode portions 28b and 29b may be located positioned on the central axis close to the midpoint thereof. Thus, electrode-supporting parts 28 and 29 as a whole may be formed convexly with expanded electrode parts 28b and 29b as the vertexes.

In addition, columnar electrodes 25 and 26 may be formed from a conductive material, such as a carbon nanotube, wherein the outer circumferential surfaces of the electrodes 25 and 26 may be fixed on expanded electrode parts 28b and 29b, respectively. Thus, electrodes 25 and 26 may be disposed so that the longitudinal direction thereof extends in the y-direction and the apical surfaces thereof face each other.

Note that in the nano-gap electrode 21 configured as described above, current may be supplied from, for example, a power source (not shown) to electrodes 25 and 26, and values of current flowing across electrodes 25 and 26 may be measured with an ammeter (not shown). Accordingly, nano-gap electrode 21 allows single-stranded DNA to be passed at least in part through nano-gap NG between electrodes 25 and 26 from the x-direction by a guiding members (not shown); an ammeter to measure the values of currents flowing across the electrodes 25 and 26 when bases of single-stranded DNA pass through the nano-gap NG between the electrodes 25 and 26; and bases constituting the single-stranded DNA to be determined on the basis of the current values.

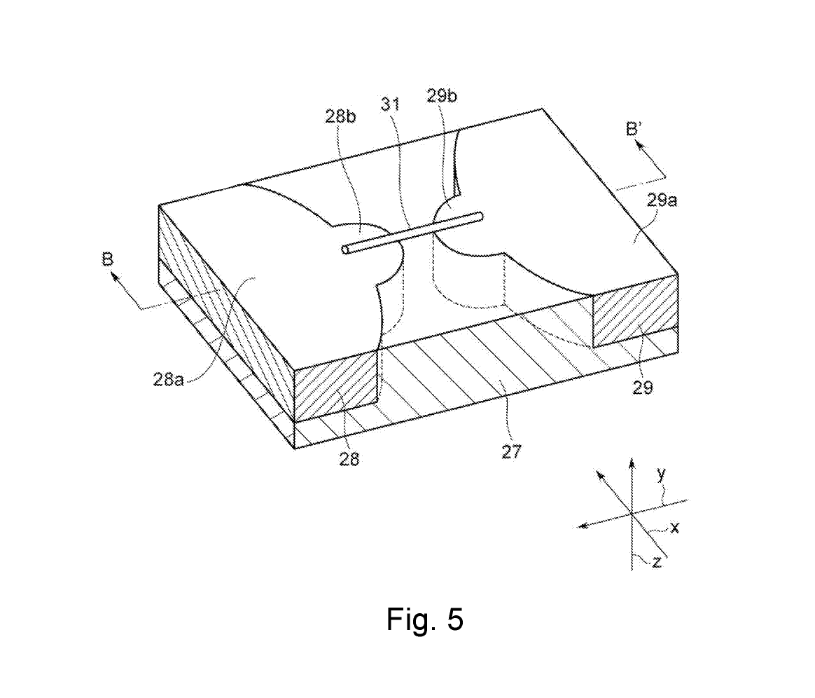

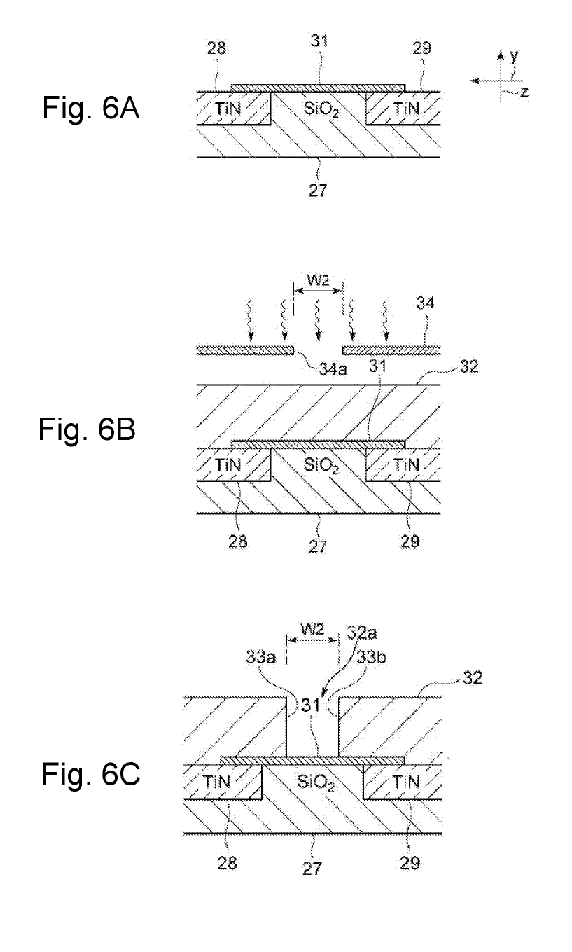

In some embodiments, a method for manufacturing a nano-gap electrode 21 may comprise producing a nano-gap NG between the electrodes 25 and 26. With reference to FIG. 5, a substrate on which electrode-supporting parts 28 and 29 having a predetermined shape may be formed adjoining silicon oxide layer 27. Then, a columnar electrode-forming part 31 may be formed from a surface of an electrode-supporting part 28 over a surface of silicon oxide layer 27 to a surface of another electrode-supporting part 29, so as to bridge over expanded electrode portions 28b and 29b of electrode-supporting parts 28 and 29. In FIG. 5, constituent elements correspond to those of FIG. 4 and are denoted by like reference numerals. FIG. 6A shows a lateral cross-sectional configuration along section B-B′ in FIG. 5.

Subsequently, as shown in FIG. 6B in which constituent elements corresponding to those of FIG. 6A are denoted by like reference numerals, a film layer of resist mask may be applied on electrode-forming part 31, silicon oxide layer 27, and electrode-supporting parts 28 and 29. Thereafter, resist mask 32 may be patterned by exposure and development using photomask 34 in which an opening 34a having a width W2 greater than width W1 of nano-gap NG as shown in FIG. 4 may be formed. Note that when resist mask 32 serving as a gap-forming mask is patterned, opening 34a is located in a region of photomask 34 at which nano-gap NG of electrode-forming part 31 is to be formed.

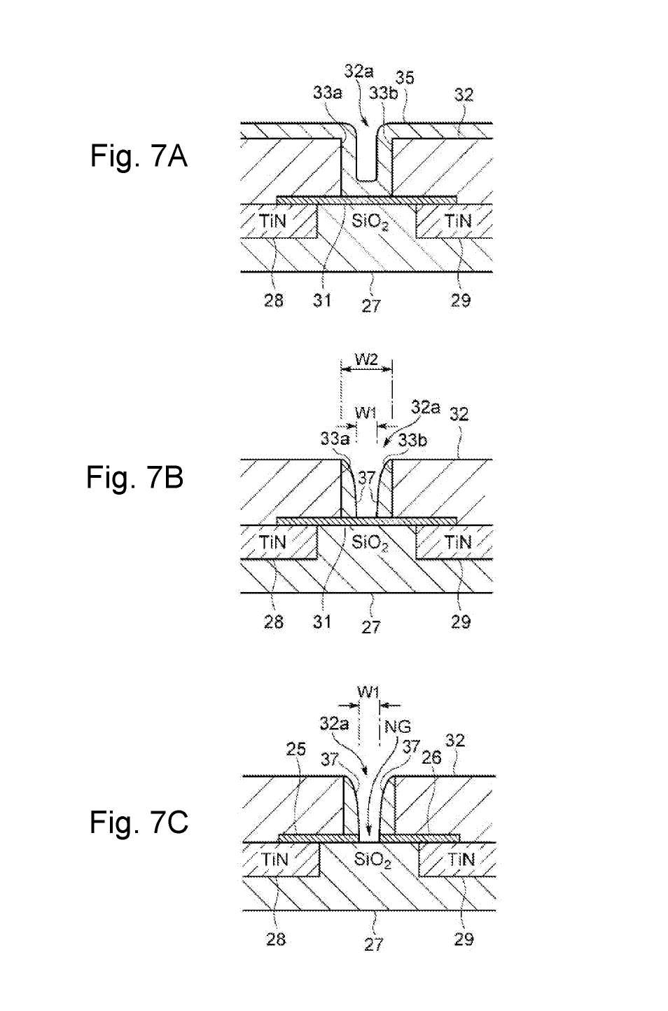

Subsequently, as shown in FIG. 6C in which constituent elements corresponding to those of FIG. 6B are denoted by like reference numerals, a gap 32a across which lateral walls 33a and 33b are disposed opposite to each other with width W2 therebetween may be formed from a region of resist mask 32 corresponding to the region at which a nano-gap NG as shown in FIG. 4 is to be formed. Thus, electrode-forming part 31 can be exposed through gap 32a. Subsequently, as shown in FIG. 7A in which constituent elements corresponding to those of FIG. 6C are denoted by like reference numerals, a sidewall-forming layer 35 which may comprise a material such as titanium (Ti) or silicon nitride (SiN), different from the material of the surfaces silicon oxide layer 27 and electrode-supporting parts 28 and 29 may be film-formed on resist mask 32 and on portions of electrode-forming part 31 and silicon oxide layer exposed within gap 32a formed from resist mask 32 by, for example, a vapor phase deposition technique, such as, for example, chemical vapor deposition (CVD). At this time, sidewall-forming layer 35, which may have a predetermined film thickness, may also be formed on lateral walls 33a and 33b of resist mask 32 within gap 32a.

Subsequently, sidewall-forming layer 35 which was film-formed on electrode-forming part 31, and silicon oxide layer 27, may be etched back within gap 32a formed from resist mask 32 by, for example, dry etching to leave sidewall-forming layer 35 along lateral walls 33a and 33b of resist mask 32. Thus, sidewalls 37 may be formed along lateral walls 33a and 33b of resist mask 32, as shown in FIG. 7B, in which constituent elements corresponding to those of FIG. 7A are denoted by like reference numerals. In some situations, sidewalls 37 may thicken gradually from the vertexes of the lateral walls 33a and 33b of resist mask 32 toward electrode-forming part 31 and silicon oxide layer 27. Accordingly, width W2 of gap 32a may be narrowed by as much as the combined thickness of both sidewalls 37. Such thickening may be used to select a nano-gap width for use in various applications, such as target molecule detection.

Consequently, the width W1 across which electrode-forming part 31 may be exposed within gap 32a may be made smaller than width W2 of gap 32a formed from resist mask 32 by as much as the film thicknesses of sidewalls 37. Subsequently, a portion of electrode-forming part 31 exposed in a W1-wide gap between sidewalls 37 disposed opposite to each other may be removed by, for example, dry etching. Thus, a nano-gap NG having a width W1 may be formed between sidewalls 37, and two electrodes 25 and 26 disposed opposite to each other across nano-gap NG may be formed, as shown in FIG. 7C, in which constituent elements corresponding to those of FIG. 7B are denoted by like reference numerals.

Width W1 through which electrode-forming part 31 may be exposed within gap 32a formed from resist mask 32 as described herein may serve as a width W1 of a nano-gap NG to be formed ultimately. Accordingly, in a process of forming sidewall-forming layer 35 on lateral walls 32a and 32b of resist mask 32, film thickness of sidewall-forming layer 35 may be selected according to a desired width W1 of a nano-gap NG. That is, when a nano-gap NG having a small width W1 is formed, sidewall-forming layer 35 may be thickly formed to decrease a width W1 of electrode-forming part 31 exposed within gap 32a formed from resist mask 32. On the other hand, when a nano-gap NG having a large width W1 is formed, sidewall-forming layer 35 may be thinly formed to increase a width W1 of electrode-forming part 31 exposed within gap 32a formed from resist mask 32.

Finally, portions of sidewalls 37 located on electrodes 25 and 26 and silicon oxide layer 27, may be removed by, for example, wet etching. Thereafter, resist mask 32 located on electrodes 25 and 26 and silicon oxide layer 27 may be removed by stripping. Thus, it is possible to form a nano-gap electrode 21 having a nano-gap NG between electrodes 25 and 26, as shown in FIG. 4. Note that in this case, the sidewalls 37 are first removed, and then the resist mask 32 is removed. Alternatively resist mask 32 may be removed first, and then sidewalls 37 may be removed.

In the above-described configuration, resist mask 32 including lateral walls 33a and 33b facing each other across a gap may be formed on electrode-forming part 31, sidewalls 37 may be respectively formed on both lateral walls 33a and 33b of resist mask 32, electrode-forming part 31 is exposed between sidewalls 37, and then electrode-forming part 31 exposed between sidewalls 37 may be removed to form a nano-gap NG.

In such a manufacturing method as described above, it is possible to form a nano-gap NG having a desired width W1 by adjusting a film thickness of each sidewall 37, in addition to a width W2 of gap 32a formed from resist mask 32. In addition, sidewalls 37 may be formed on lateral walls 33a and 33b formed from resist mask 32 in this manufacturing method, and therefore, a width W2 of gap 32a formed from resist mask 32 may be made smaller by as much as the film thicknesses of sidewalls 37. Thus, it is possible to form a nano-gap NG having a width W1 even smaller than a width W2 of gap 32a formed in the patterned resist mask 32.

According to the above-described configuration, a nano-gap NG having a width W1 adjusted by the film thicknesses of sidewalls 37 may be formed on electrode-forming part 31 using sidewalls 37 disposed on electrode-forming part 31 as a part of a mask. Consequently, it is possible to form not only a nano-gap NG that is the same in width W1 as a conventional nano-gap, but also to form a nano-gap NG that is even smaller in width W1 than a conventional nano-gap formed using conventional lithographic techniques.

In some cases, resist mask 32 having a gap 32a may be directly formed on electrode-forming part 31. In other embodiments, an electrode-forming part, on a surface on which a hard mask may be formed, may be used to form a gap-forming mask having a gap in the hard mask, and a gap-forming mask may be disposed on an electrode-forming part in a gap formed by the hard mask.

In this embodiment, only hard mask material exposed between sidewalls 37 formed on both lateral walls 33a and 33b formed from resist mask 32 may be removed to form a gap in the hard mask. Then, a portion of electrode-forming part 31 through a gap in the hard mask located between sidewalls 37 may be removed by, for example, dry etching, thereby forming a nano-gap NG between sidewalls 37.

Also as described herein, a resist mask 32 may be applied as a mask. In other embodiments, a mask made from one of various materials other than a resist may be applied, as long as a gap can be formed and sidewalls can be formed on the lateral walls of this gap. Note that a nano-gap electrode to be ultimately manufactured may be one in which sidewalls 37 may be left in place rather than being removed, as shown in FIG. 7C. Alternatively, sidewalls may be removed as part of a subsequent process. In some embodiments, resist mask 32 may be left in place; as an alternative, resist mask 32 may be removed.

Described herein are alternative methods for manufacturing nano-gap electrode 21 shown in FIG. 4. In some embodiments, a substrate on which the electrode-supporting parts 28 and 29 which may have a predetermined shape may be formed adjacent silicon oxide layer 27 may be prepared first. Then, an electrode-forming part 31 made of a carbon nanotube may be formed or applied from a surface of one electrode-supporting part 28 over a surface of silicon oxide layer 27 to a surface of another electrode-supporting part 29, so as to bridge over expanded electrode portions 28b and 29b of electrode-supporting parts 28 and 29, as shown in FIG. 5.

In other embodiments, electrode-forming part 31 may comprise a gold, Pt or other metal or alloy nanowires, or may comprise a semiconductor nanowires, wherein a nanowires may have a diameter of a nanometer, or may have a diameter as large as several nanometers or larger.

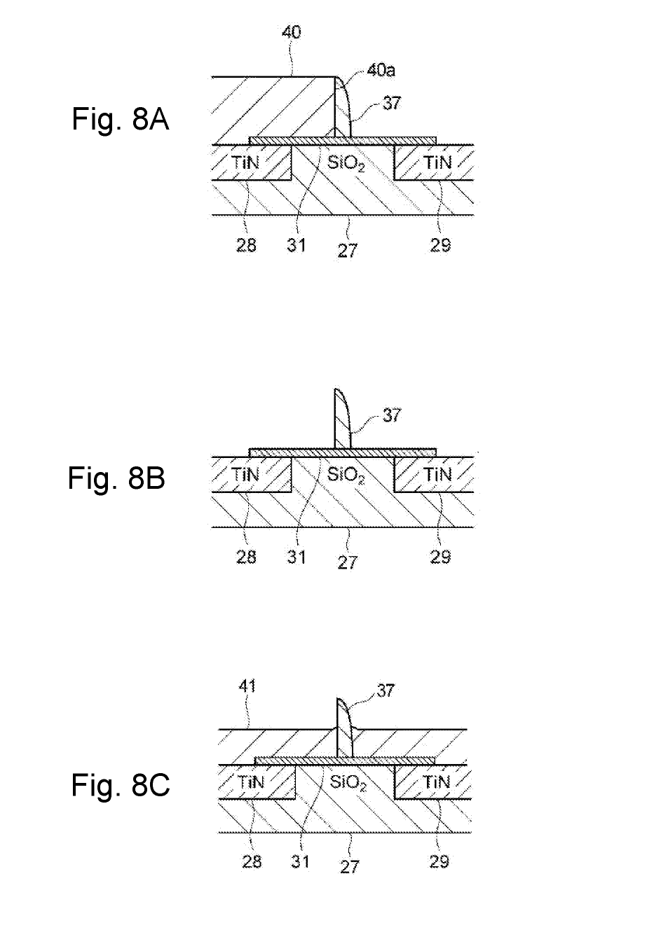

In other embodiments, electrode forming part 31 may comprise a thin layer (e.g., a monolayer) of a metal or alloy or semiconductor. Subsequently, a layer of sidewall-forming mask 40 made from, for example, a resist material, may be formed as a film on electrode-forming part 31 and silicon oxide layer 27. Thereafter, sidewall-forming mask 40 may be patterned using a photolithographic technique. Consequently, as shown in FIG. 8A which shows a lateral cross-sectional configuration of section B-B′ in FIG. 5, a lateral wall 40a of a sidewall-forming mask 40 may be formed on electrode-forming part 31 and silicon oxide layer 27 in alignment with a region at which a nano-gap NG of electrode-forming part 31 as shown in FIG. 4 is to be formed.

Subsequently, a sidewall-forming layer (not shown) may be formed as a film on sidewall-forming mask 40 and exposed portions of electrode-forming part 31 and silicon oxide layer 27 which may comprise a material, such as titanium (Ti) or silicon nitride (SiN), different from the material of electrode-forming part 31. Thereafter, sidewall-forming layer may be etched back by dry etching to leave a portion of sidewall-forming layer along lateral wall 40a of sidewall-forming mask 40. Thus, a sidewall 37 may be formed along lateral wall 40a of sidewall-forming mask 40, as shown in FIG. 8A. Note that sidewall 37 formed in this way may thicken gradually from the vertex of lateral wall 40a of sidewall-forming mask 40 toward electrode-forming part 31 and silicon oxide layer 27. Accordingly, a maximum thickness of sidewall 37 can be a width W1 of a nano-gap NG to be formed ultimately.

Subsequently, as shown in FIG. 8B in which constituent elements corresponding to those of FIG. 8A are denoted by like reference numerals, sidewall-forming mask 40 may be removed to leave sidewall 37 built vertically on electrode-forming part 31. The sidewall in such a case can be a free-standing sidewall. The free-standing sidewall can have a width that is less than or equal to about 10 nanometers (nm), 5 nm, 4 nm, 3 nm, 2 nm, 1 nm, 0.9 nm, 0.8 nm, 0.7 nm, 0.6 nm or 0.5 nm. With reference to FIG. 8C, in which constituent elements corresponding to those of FIG. 8B are denoted by like reference numerals, a resist mask 41 which may serve as a gap-forming mask may be formed on electrode-forming part 31 and silicon oxide layer 27. Such a resist mask 41 as described above may be formed by coating a resist coating material on exposed portions of electrode-forming part 31 and silicon oxide layer 27 and hardening the resist coating material. Here, the resist coating material may be selected to form resist mask 41 may be low in viscosity. Accordingly, even if the resist coating material adheres to the upper portion of sidewall 37 when coated on, for example, electrode-forming part 31 and silicon oxide layer 27, the material drops off the upper portion of the sidewall 37 due to the weight of the material itself, and centrifugal force and the like when centrifugally formed into a uniform film. Thus, the upper portion of sidewall 37 may be exposed without being buried in the resist coating material. Consequently, the upper portion of sidewall 37 may be exposed out of a surface of resist mask 41.

Note that if the viscosity of the resist coating material is high and any portion thereof adhering to the upper portion of sidewall 37 hardens thereon, and therefore, sidewall 37 as a whole is covered with the resist mask 41, or if the resist mask 41 has a large film thickness, and therefore, sidewall 37 as a whole is covered with the resist mask 41, the upper portion of sidewall 37 may be exposed out of the surface of resist mask 41 by etching back the resist mask 41, as shown in FIG. 8C.

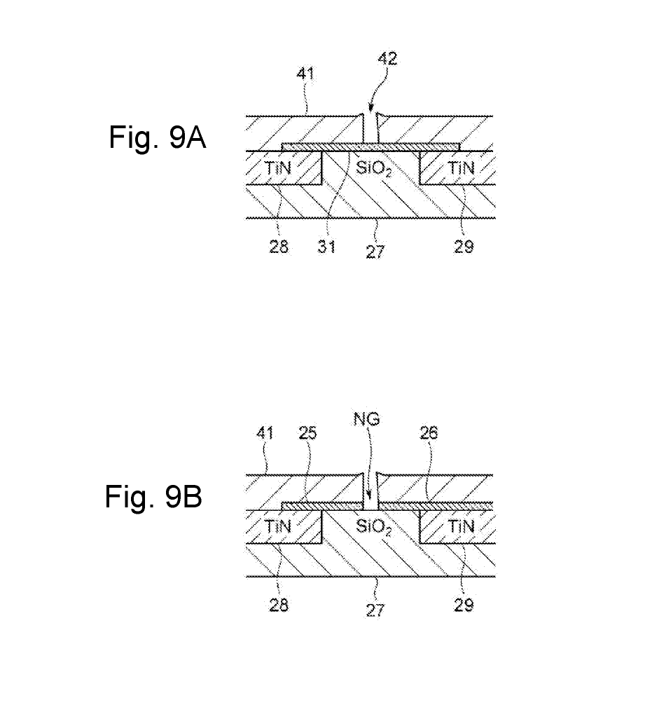

Subsequently, as shown in FIG. 9A in which constituent elements corresponding to those of FIG. 8C are denoted by like reference numerals, sidewall 37, an upper portion of which may be exposed, may be removed by, for example, wet etching, to form a gap 42 in a region of resist mask 41 in which sidewall 37 was located. Thus, electrode-forming part 31 may be exposed through gap 42. Then, as shown in FIG. 9B in which constituent elements corresponding to those of FIG. 9A are denoted by like reference numerals, a portion of electrode-forming part 31 exposed through gap 42 of resist mask 41 may be removed by, for example, dry etching, thereby forming a nano-gap NG wherein electrodes 25 and 26 disposed opposite to each other across nano-gap NG on electrode-forming part 31.

The width across which electrode-forming part 31 may be exposed through gap 42 of resist mask 41 as described herein serves as a width W1 of nano-gap NG as shown in FIG. 4 which will be formed subsequently. Accordingly, in a process of forming a sidewall-forming layer on lateral wall 40a of sidewall-forming mask 40, a film thickness of a sidewall-forming layer may be selected according to a desired width W1 of a nano-gap NG. That is, when a nano-gap NG having a small width W1 is formed, a sidewall-forming layer may be thinly formed to decrease the width of electrode-forming part 31 exposed through gap 42 of resist mask 41. On the other hand, when a nano-gap NG having a large width W1 is formed, a sidewall-forming layer may be thickly formed to increase the width of electrode-forming part 31 exposed through gap 42 of resist mask 41.

Finally, resist mask 41 located on electrodes 25 and 26 and silicon oxide layer 27 may be removed by, for example, stripping. Thus, it is possible to form a nano-gap electrode 21 having a nano-gap NG between electrodes 25 and 26, as shown in FIG. 4. In other embodiments, resist mask 41 may be left in place, and may, for example, be used as a channel through which DNA may move so as to interact with electrodes 25 and 26.

In the above-described configuration, sidewall 37 may be formed on lateral wall 40a of sidewall-forming mask 40 disposed on electrode-forming part 31, and then sidewall-forming mask 40 may be removed to vertically build sidewall 37. Resist mask 41 may be formed so as to surround sidewall 37. Then, sidewall 37 surrounded by resist mask 41 may be removed to form gap 42 in resist mask 41 and expose the electrode-forming part 31 through gap 42. Thereafter, any portion(s) of electrode-forming part 31 exposed through gap 42 may be removed to form a nano-gap NG within gap 42.

In such a manufacturing method as described herein, a width of gap 42 to be formed in resist mask 41 may be adjusted by adjusting a film thickness of each sidewall 37. Consequently, a nano-gap NG to be formed within gap 42 may be formed to a desired width W1. In addition, since sidewall 37 may be formed to with extremely small film thickness, it is possible to form a nano-gap NG having an extremely small width W1 corresponding to the thickness of sidewall 37.

According to the above-described configuration, a nano-gap NG having a width W1 adjusted by the film thicknesses of sidewalls 37 may be formed on electrode-forming part 31 using sidewall 37 disposed on electrode-forming part 31 as a mask. Consequently, it is possible to form not only a nano-gap NG that is the same in width W1 as a conventional nano-gap, but also to form a nano-gap NG that is even smaller in width W1 than the conventional nano-gap.

Note that as described herein above wherein a sidewall-forming layer is made to remain along lateral wall 40a of sidewall-forming mask 40 to form sidewall 37 may be built vertically into a wall shape. In other embodiments, only the sidewall-forming layer on sidewall-forming mask 40 may be removed to leave a sidewall-forming layer along lateral wall 40a of sidewall-forming mask 40. In addition, a sidewall-forming layer may be made to remain on silicon oxide layer 27 and electrode-forming part 31 where sidewall-forming mask 40 is not present. Thus, there may be formed a sidewall having a bottom surface with an L-shape in cross section.

Sidewall-forming mask 40 and resist mask 41 serving as a gap-forming mask may be formed from a resist material. In other embodiments sidewall-forming mask(s) and gap-forming mask(s) may be formed from various other materials.

The present disclosure provides methods for manufacturing a nano-gap electrode 21 as shown in FIG. 4. Note that a description of the configuration of the nano-gap electrode 21 shown in FIG. 4 will be omitted here to avoid duplicating the previous description. In some embodiments, a substrate on which electrode-supporting parts 28 and 29 having a predetermined shape are formed adjacent silicon oxide layer 27 may be prepared first. Then, an electrode-forming part 31 made of a carbon nanotube may be formed from a surface of one electrode-supporting part across a surface of silicon oxide layer 27 to a surface of another electrode-supporting part 29, so as to bridge over expanded electrode parts 28b and 29b of electrode-supporting parts 28 and 29, as shown in FIG. 5.

In addition, an etch-stop film (not shown) which may be made from, for example, silicon nitride (SiN) may be formed on electrode-forming part 31 and silicon oxide layer 27 wherein, in order to prevent electrode-forming part 31, which may be comprise a carbon nanotube, from being etched in the later-described course of manufacture in which a sidewall may be removed by wet etching.

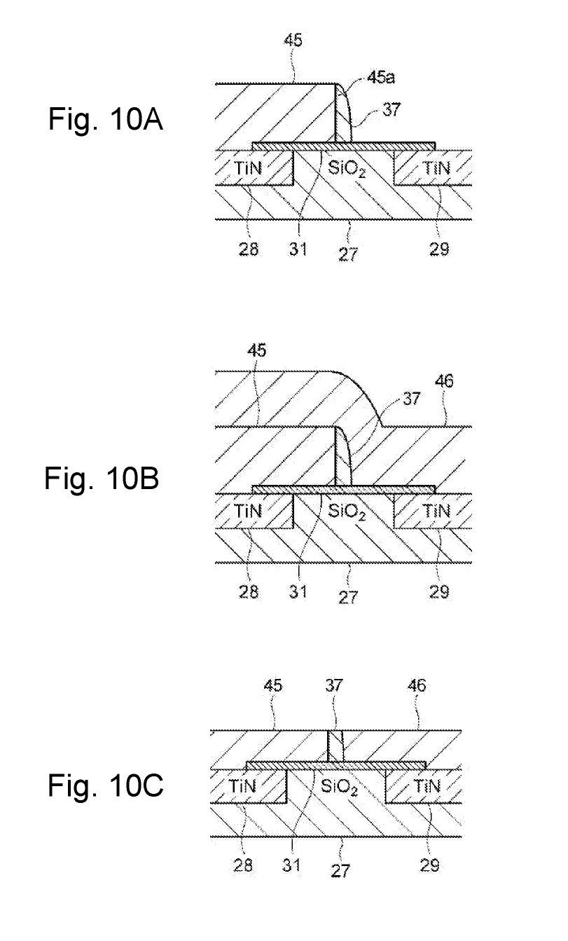

Subsequently, a layer-like first gap-forming mask which may be made from, for example, polysilicon or amorphous silicon may be formed as a film on an etch-stop film on electrode-forming part 31 and silicon oxide layer 27 by a CVD method or the like. Thereafter, first gap-forming mask may be patterned using a photolithographic technique. Consequently, as shown in FIG. 10A which depicts a method of fabricating a device with a lateral cross-sectional view of section B-B′ in FIG. 5, a lateral wall 45a of a first gap-forming mask 45 may be formed on an etch-stop film (not shown) which may be located on electrode-forming part 31 and silicon oxide layer 27 in alignment with a region where a nano-gap NG of electrode-forming part 31 as shown in FIG. 4 may be formed.

Subsequently, a sidewall-forming layer (not shown) which may be made from, for example, silicon oxide which may be a material different from the material of electrode-forming part 31 may be formed as a film on an etch-stop film on electrode-forming part 31 and silicon oxide layer 27 and first gap-forming mask 45. Thereafter, sidewall-forming layer may be etched back by dry etching to leaving a sidewall-forming layer along lateral wall 45a of first gap-forming mask 45. Thus, a sidewall 37 may be formed along lateral wall 45a of first gap-forming mask 45, as shown in FIG. 10A. Note that sidewall 37 formed in this way may thicken gradually from the vertex of lateral wall 45a of first gap-forming mask 45 toward electrode-forming part 31 and silicon oxide layer 27 and an etch-stop film. Accordingly, a maximum thickness of sidewall 37 may be a width W1 of a nano-gap NG to be formed subsequently.

Subsequently, as shown in FIG. 10B in which constituent elements corresponding to those of FIG. 10A are denoted by like reference numerals, a second gap-forming mask 46 which may be made from, for example, polysilicon or amorphous silicon may be formed as a film on an etch-stop film (not shown) located on electrode-forming part 31 and silicon oxide layer 27, on sidewall 37 and on first gap-forming mask 45 by a CVD method or the like.

Then, regions of second gap-forming mask 46 covering first gap-forming mask 45 and sidewall 37, first gap-forming mask 45 and sidewall 37 may be polished and may be over-polished by planarization processing, such as CMP. Thus, surfaces of first gap-forming mask 45, sidewall 37 and second gap-forming mask 46 may be exposed, as shown in FIG. 10C in which constituent elements corresponding to those of FIG. 10B are denoted by like reference numerals.

In some embodiments, a largely inclined upper region of the side surface of the sidewall 37 may be polished and first gap-forming mask 45, sidewall 37, and second gap-forming mask 46 may be polished, and may be over-polished in a planarization processing operation until a cross section of sidewall 37 between first gap-forming mask 45 and second gap-forming mask 46 may be formed into a substantially quadrilateral shape. Note that in some embodiments only regions of second gap-forming mask 46 covering first gap-forming mask 45 and sidewall 37 may be polished, as long as surfaces of first gap-forming mask 45, sidewall 37, and second gap-forming mask 46 can be exposed when a planarization processing operation is performed.

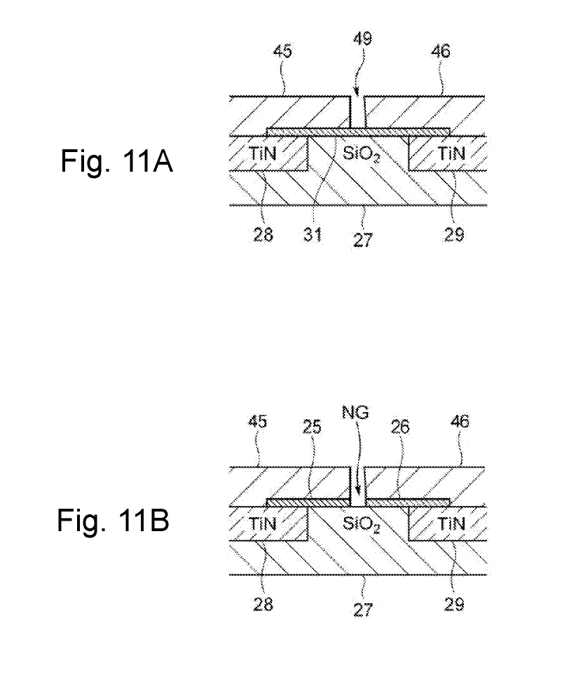

Subsequently, as shown in FIG. 11A, in which constituent elements corresponding to those of FIG. 10C are denoted by like reference numerals, sidewall 37 located between first gap-forming mask 45 and second gap-forming mask 46 may be removed by, for example, wet etching to form a gap 49 that is the same width as sidewall 37. Thus, an etch-stop film (not shown) on electrode-forming part 31 may be exposed through gap 49.

Then, as shown in FIG. 11B, in which constituent elements corresponding to those of FIG. 11A are denoted by like reference numerals, portions of an etch-stop film (not shown) and electrode-forming part 31 exposed through gap 49 between first gap-forming mask and second gap-forming mask 46 may be removed by, for example, dry etching, thereby forming a nano-gap NG and electrodes 25 and 26 disposed oppositely to each other across a nano-gap NG in electrode-forming part 31.

The width of electrode-forming part 31 within gap 49 located between first gap-forming mask 45 and second gap-forming mask 46 as described above serves as a width W1 of nano-gap NG as shown in FIG. 4 to be formed subsequently. Accordingly, in a process of forming a sidewall-forming layer on lateral wall 45a of first gap-forming mask 45, a film thickness of a sidewall-forming layer may be selected according to a desired width W1 of a nano-gap NG. That is, when a nano-gap NG having a small width W1 is formed, a sidewall-forming layer may be thinly formed to decrease the width of electrode-forming part 31 exposed within gap 49 between first gap-forming mask 45 and second gap-forming mask 46. On the other hand, when a nano-gap NG having a large width W1 is formed, a sidewall-forming layer may be thickly formed to increase the width of electrode-forming part 31 exposed within gap 49 between first gap-forming mask 45 and second gap-forming mask 46.

Finally, first gap-forming mask 45 and second gap-forming mask 46, located on electrodes 25 and 26 and silicon oxide layer 27, may be removed by, for example, wet etching. Thus, it is possible to form a nano-gap electrode 21 having a nano-gap NG between electrodes 25 and 26, as shown in FIG. 4.

In the above-described configuration, sidewall 37 may be formed on lateral wall 45a of first gap-forming mask 45 disposed on electrode-forming part 31, and then second gap-forming mask 46 may be formed so as to abut on sidewall 37. Thus, sidewall 37 may be disposed between first gap-forming mask 45 and second gap-forming mask 46. Then, surfaces of first gap-forming mask 45, sidewall 37, and second gap-forming mask 46 may be exposed, and sidewall 37 may be removed to form gap 49 between first gap-forming mask 45 and second gap-forming mask 46. Thus, a nano-gap NG may be formed by removing a portion of electrode-forming part 31 within gap 49.

In such a manufacturing method as described herein, it is possible to form a nano-gap NG having a desired width W1 by adjusting a film thickness of sidewall 37. In addition, sidewall 37 may be formed with an extremely small film thickness. It is therefore possible to form a nano-gap NG having an extremely small width W1 corresponding to the thickness of sidewall 37. In addition, unlike in a conventional manufacturing method, this manufacturing method does not require patterning a metal mask when forming a nano-gap NG. It is therefore possible to form a nano-gap NG without undue effort.

According to the above-described configuration, a nano-gap NG having a width W1 adjusted by a film thickness of sidewall 37 may be formed in electrode-forming part 31 using sidewall 37 disposed on electrode-forming part 31 as a mask. Consequently, it is possible to form not only a nano-gap NG that is the same width W1 as a conventional nano-gap, but also to form a nano-gap NG that is even smaller in width W1 than a conventional nano-gap.

In some cases, second gap-forming mask 46 may be directly formed on first gap-forming mask 45, as shown in FIG. 10B. In other embodiments, a first gap-forming mask 45 on a surface on which a hard mask is formed may be used without directly forming second gap-forming mask 46 on first gap-forming mask 45. Even in this case, it is possible to dispose sidewall 37 between first gap-forming mask 45 and second gap-forming mask 46. Consequently, it is possible to form gap 49 between first gap-forming mask 45 and second gap-forming mask 46 by removing sidewall 37.

It should be noted that the present invention is not limited to the present embodiments, but may be modified and carried out in various other ways within the scope of the subject matter of the present invention. For example, various materials may be used as the materials of electrodes 5 and 6 (25 and 26), substrate 2, silicon oxide layer 4 (27) sidewall 11 (37), and the like. In addition, first electrode-forming part 9, second electrode-forming part 12, and electrodes 5 and 6 may have various shapes. Likewise, electrode-forming part 31 and electrodes 25 and 26 may have various shapes.

For example, although electrode-forming part 31 is described as being made of a carbon nanotube, the present invention is not limited to these embodiments. For example, an electrode-forming part may be formed from a metal material having one of various other shapes, including simple rectangular solid and columnar shapes.

Here, a description will be made of a manufacturing method as described in association with the descriptions of FIGS. 6 and 7. If, for example, an electrode-forming part made from a rectangular solid-shaped metal material is applied as an electrode-forming part, a resist mask 32 having an opening 32a may be disposed on rectangular solid-shaped electrode-forming part(s), sidewalls 37 may be formed along both lateral walls 33a and 33b of resist mask 32, and a portion of electrode-forming part exposed between sidewalls 37 may be removed. Thus, it is possible to form a nano-gap NG between sidewalls 37 and rectangular solid-shaped electrodes disposed opposite to each other across a nano-gap NG.

With reference to FIGS. 6-11, the electrode-supporting parts 28 and 29 may be formed adjacent to silicon oxide layer 27 on a substrate and electrode-forming part 31 may be disposed on surfaces of electrode-supporting parts 28 and 29. Alternatively, an electrode-forming part having various shapes may be disposed on a substrate in which electrode-supporting parts 28 and 29 are not disposed adjacent silicon oxide layer 27 on a substrate, but may be provided simply with a silicon oxide layer or may comprise only of a silicon substrate. Alternatively, an electrode-forming part may be disposed on a substrate, and electrode-supporting parts may be protrudingly formed on upper portions of an electrode-forming part on both sides thereof. Thus, embodiments may have a configuration in which an electrode-forming part is located between two electrode-supporting parts disposed so as to face each other on a substrate.

In addition, in the above-described embodiments, a description has been made of a nano-gap electrode 1 (21) in which single-stranded DNA may be passed at least in part through a nano-gap NG between electrodes 5 and 6 (25 and 26), and values of current(s) flowing across the electrodes 5 and 6 (25 and 26) when bases of single-stranded DNA pass through a nano-gap NG between electrodes 5 and 6 (25 and 26) may be measured with an ammeter. The present invention is not limited to these embodiments, however. The nano-gap electrode may be used in various other applications. In some embodiments, the nano-gap may be utilized for double stranded DNA, and my therefore be fabricated to have a different dimension which may be more suitable for measurement of double stranded DNA. In other embodiments, the nano-gap may be utilized for other biomolecules, such as amino acids, lipids, or carbohydrates, and may thus be fabricated with a width appropriate for each type of biomolecule.

In the description accompanying FIGS. 6-11, methods have been described in which sidewall 11 or 37 may be formed so as to thicken gradually from the vertex of a lateral wall toward silicon oxide layer 27 may be applied as the sidewall. In other embodiments, a sidewall-forming layer, differing in film thickness depending on a location of film formation, may be formed under various film-forming conditions (temperature, pressure, gas used, flow ratio, and the like), without forming a film on a sidewall in a conformal manner. Thus, there may be a film applied to a sidewall formed so as to gradually thin from the vertex toward a silicon oxide layer, or a sidewall the width of which may have a maximum width at an intermediate location between the vertex and a silicon oxide layer or at various other locations.

The present disclosure provides a method for manufacturing the nano-gap electrode 1 having a nano-gap NG between electrodes 5 and 6. Substrate 2 for which the silicon oxide layer 4 may be formed on a silicon substrate 3 may be prepared first. Subsequently an electrode forming layer 79 may be added and a first mask 72 made from, for example, silicon nitride (SiN) and having a lateral wall 72a may be formed on a predetermined region of electrode forming layer 79 using a photolithographic technique.

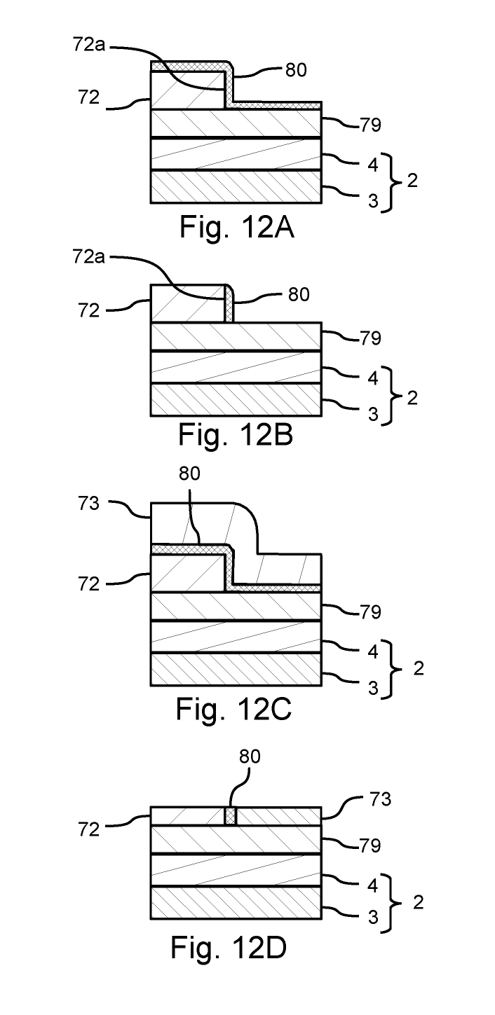

Subsequently, as shown in FIG. 12A, a sidewall-forming layer 80 made from a material, such as titanium (Ti) different from the material of the surface (which may comprise titanium nitride) of electrode forming layer 79 may be formed as a film on electrode-forming part 79 and exposed portions of substrate 2 by, for example, a chemical vapor deposition (CVD) technique. At this time, a sidewall-forming layer 80 may be formed along lateral wall 72a of first mask 72. The film thickness of sidewall-forming layer 80 to be formed on lateral wall 72a may be selected according to a desired width W1 of nano-gap NG. That is, when a nano-gap NG having a small width W1 is formed, sidewall-forming layer 80 may be formed with a small film thickness. On the other hand, when a nano-gap NG having a large width W1 is formed, sidewall-forming layer 80 may be formed with a large film thickness.

Subsequently, as shown in FIG. 12B a sidewall-forming layer 80 film-formed on first mask 72 and exposed portions of the electrode forming layer 79, may be etched by, for example, dry etching to leave a portion of sidewall-forming layer 80 along lateral wall 72a of the first mask 72. The etching process may be configured to be perpendicular with respect to substrate 2, or may be angled such that a portion of sidewall-forming layer 80 may be at least partially protected from etching by lateral wall 72a of first mask 72.

Subsequently, as shown in FIG. 12C a second mask 73 may be deposited by, for example, a sputtering method.

Subsequently, as shown in FIG. 12D first mask 72 and sidewall forming layer 80, as well as regions of second mask 73 may be polished or may be over polished by planarization processing, such as CMP (Chemical and Mechanical Polishing).

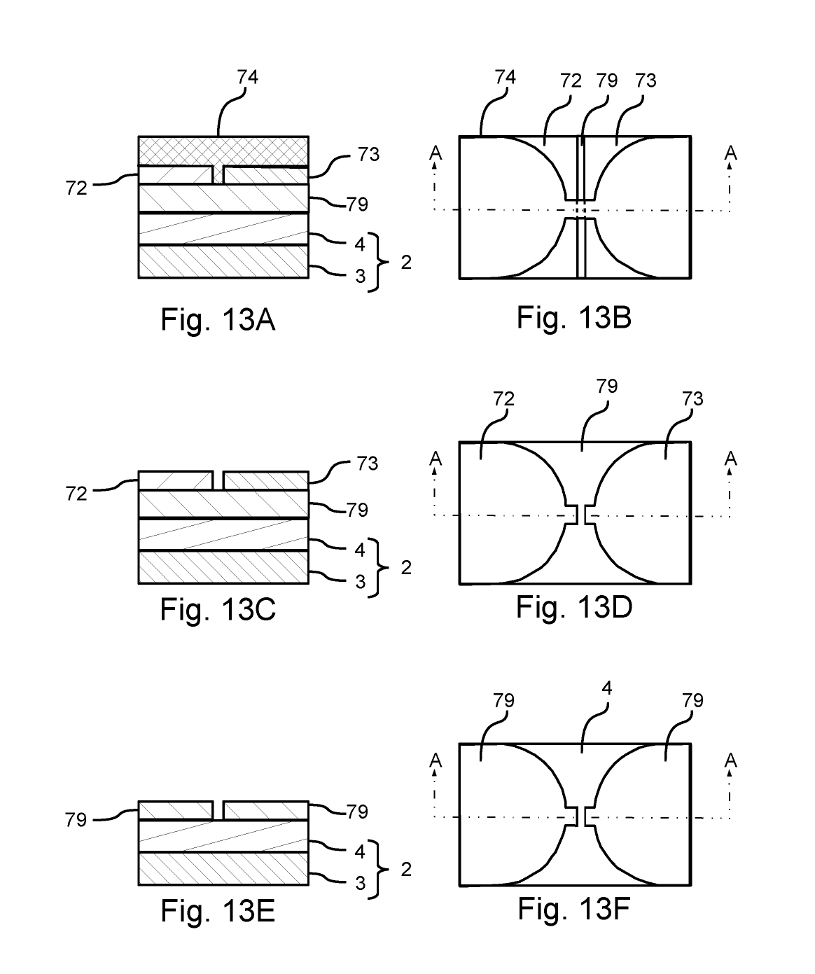

Subsequently, as shown in FIG. 13A (center cross section view) and FIG. 13B (top view) a layer of resist may be applied and patterned. Portions of first mask 72 and second mask 73 left exposed by patterned resist 74 may then be etched away. Patterned resist 74 may then be removed exposing remaining mask layers as shown in FIG. 13C (center cross section view) and FIG. 13D (top view). Remaining first mask 72 and remaining second mask 73 may then be used to etch electrode forming layer 79, and may subsequently be removed, as shown in FIG. 13E (center cross section view) and FIG. 13F (top view) creating a structure as shown in FIG. 1.

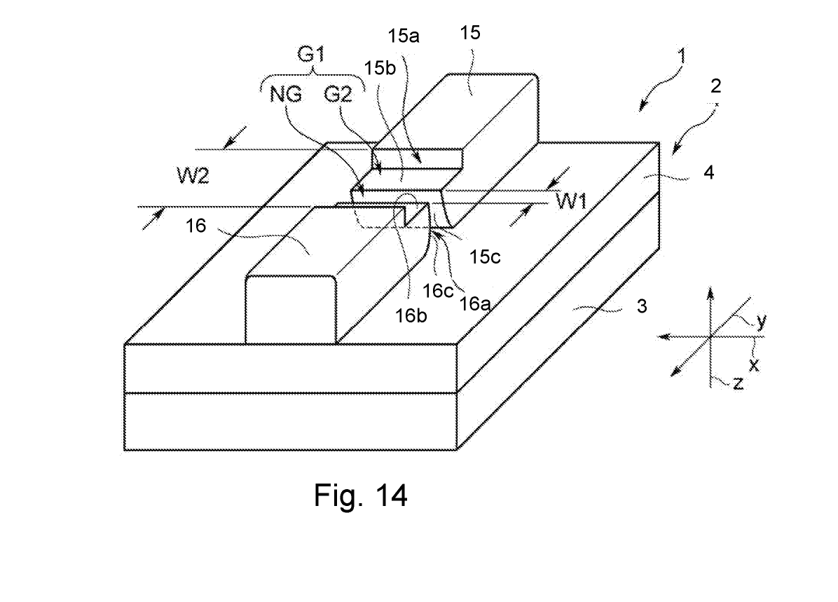

In FIG. 14, reference numeral 1 denotes a nano-gap electrode according to a one embodiment of the present invention. In this nano-gap electrode 1, opposing electrodes 15 and 16 may be disposed on a substrate 2. A hollow gap G1 with a minimum width W1 which may be nanoscale (e.g., no larger than 1000 nm), may be formed between these electrodes 15 and 16. The substrate 2 may comprise, for example, a silicon substrate 3 and a silicon oxide layer 4 formed thereon. The substrate 2 may thus have a configuration in which two electrodes 15 and 16 which form a pair may be formed on a silicon oxide layer 4.

In some embodiments, the gap G1 formed between the electrodes 15 and 16 may comprise a mask width gap G2 and a nano-gap NG narrower than the width W2 corresponding to mask width gap G2. The nano-gap electrode 1 of the present invention is characterized in that it is possible to form a nano-gap NG narrower than the width W2 of a mask width gap G2 formed with a mask used in the course of manufacture (described later). In some embodiments, the nano-gap NG may be formed with a minimum width W1 of from 0.1 nm to 30 nm, or a width W1 no greater than 10 nm, no greater than 5 nm, no greater than 2 nm, no greater than 1 nm, or no greater than 0.5 nm, or a width W1 of from 1.5 nm to 0.3 nm, or from 1.2 nm to 0.5 nm, or from 0.9 nm to 0.65 nm, or from 1.2 nm to 0.9 nm, or from 1.0 nm to 0.8 nm, or from 0.8 nm to 0.7 nm. The widths as described herein may utilized for the gap spacing for any of the nano-gaps described herein.

In practice, each of these electrodes 15 and 16 may be formed from one of various types of metal silicides, including titanium silicide, molybdenum silicide, platinum silicide, nickel silicide, cobalt silicide, palladium silicide, and niobium silicide or combinations thereof, or alloys of silicides with other materials, or may be silicides which may be doped with various materials as my be commonly used for doping of semiconductors. The electrodes 15 and 16 may have the same configuration and may be formed bilaterally symmetrically across a nano-gap NG on the substrate 2. Sidewalls 15a and 16a at respective ends of the electrode parts 15 and 16 may be disposed opposite to each other across the nano-gap NG. In practice, in some embodiments, the electrodes 15 and 16 may be composed of rectangular solids, the longitudinal cross section of which may be quadrilateral and the longitudinal direction of which may extend in a y-direction. The electrodes 15 and 16 may be disposed so that the long-side central axes thereof are positioned on the same y-axis straight line, and so that the front surfaces of the sidewalls 15a and 16a face each other.

Shoulders 15b and 16b may comprise L shaped recesses, which may be formed into the upper corners of the sidewalls 15a and 16a of the electrodes 15 and 16. In addition, trailing curved surfaces 15c and 16c increasingly gently recess corresponding to increased downward distance from the bottom surfaces of shoulders 15b and 16b formed in the sidewalls 15a and 16a. Thus, a quadrilateral mask width gap G2 bridging over the electrodes 15 and 16 and the gap there between may be formed between shoulders 15b and 16b. Consequently, a nano-gap NG is formed between the curved surfaces 15c and 16c corresponding to the distance between the ends of the electrodes, which increasingly widens closer to the substrate 2.

In other embodiments, the surface above the shoulders 15b and 16b forming the mask width gap G2 may be removed by polishing by, for example, CMP, so as to leave only the nano-gap NG between the electrodes 15 and 16.

Note that in a nano-gap electrode 1 configured as described above, current can be supplied from, for example, a power source (not shown) to the electrodes 15 and 16, and the values of current flowing across the electrodes 15 and 16 may be measured with an ammeter (not shown). Accordingly, a nano-gap electrode 1 allows single-stranded DNA to pass through a nano-gap NG between electrodes 15 and 16 from an x-direction orthogonal to the y-axis, which may be the longitudinal axis of the electrodes 15 and 16, and/or from a z-direction, which may be the height axis of the electrodes 15 and 16, and intersects at right angles with the y-axis; an ammeter may be utilized to measure the values of current flowing across electrodes 15 and 16 when bases of single-stranded DNA pass through the nano-gap NG between the electrodes 15 and 16; and bases comprising a single-stranded DNA may be determined on the basis of the current values.

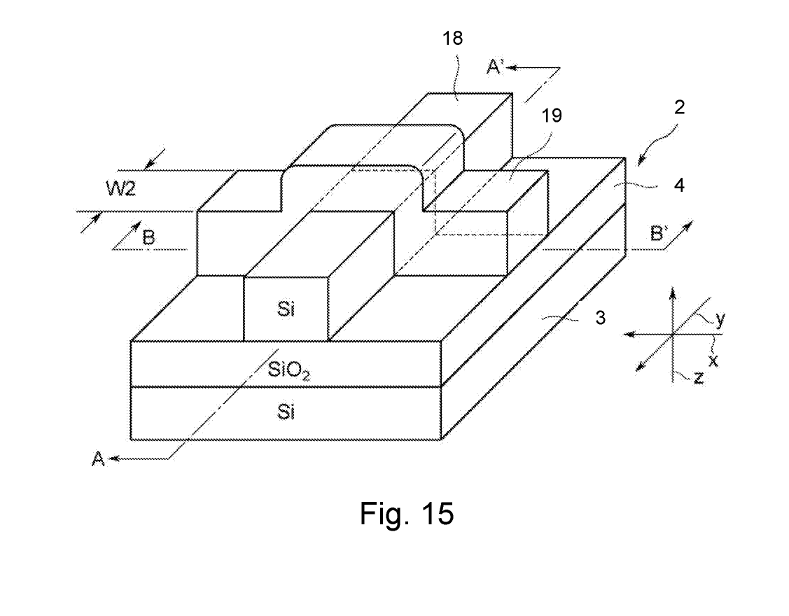

In some embodiments, a method for manufacturing a nano-gap electrode 1 as described above may comprise a method wherein a substrate 2 whereby a layer which may be a silicon oxide layer 4 may be formed on a substrate which may be a silicon substrate 3 may be prepared as shown in FIG. 15. Then, an electrode-forming part 18, which may be rectangularly shaped, and which may be made from silicon and may have a longitudinal axis extending in the y-axis may be formed on the silicon oxide layer 4 using a lithographic technique. Subsequently, a mask layer 19 (not shown) which may be made from silicon nitride (SiN) may be formed as a film on substrate 2 and electrode-forming part 18; this mask layer 19 may be formed using a resist mask, which may be patterned by standard lithographic processes.

Consequently, a mask layer 19, which may have rectangular cross section, and which may be made from silicon nitride (SiN) may be formed so as to bridge over the electrode-forming part 18 along the x-axis orthogonal to the y-axis, which may be the longitudinal axis of electrode-forming part 18. Note that width W2 of mask layer 19 serves to form mask width G2 between electrodes 15 and 16 when electrodes 15 and 16 may be formed. In some embodiments it may therefore be desirable to change the method of patterning of the resist mask so as to select the width W2 of mask layer 19, which may require a method which minimizes the width of the resist mask corresponding to the width W2 of mask layer 19.

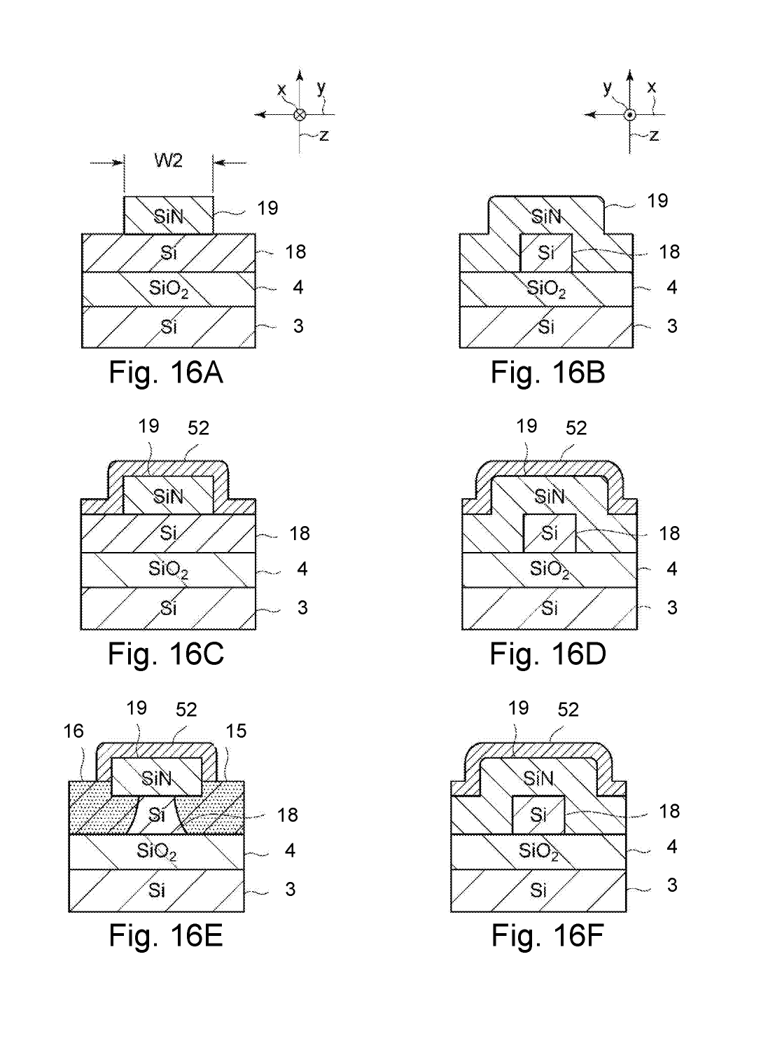

Here, attention will be focused on the structures illustrated in cross sections A-A′ and B-B′ in FIG. 15 to describe a process of manufacturing nano-gap electrode 1. FIG. 16A shows the structure of cross section A-A′ in FIG. 15, whereas FIG. 16B shows the structure of cross section B-B′ in FIG. 15. As shown in FIG. 16C, in which constituent elements corresponding to those of FIG. 16A are denoted by like reference numerals, and FIG. 16D in which constituent elements corresponding to those of FIG. 16B are denoted by like reference numerals, a silicide-generating layer 52, which may be made from a metal element, such as titanium, molybdenum, platinum, nickel, cobalt, palladium or niobium, may be formed as a film on mask layer 19 and electrode-forming part 18 by, for example, sputtering. Note that at this time, silicide-generating layer 52 may also be formed as a film on substrate 2 which may be exposed in regions not covered by mask layer 19 and electrode-forming part 18.

Subsequently, a heat treatment may be performed to react electrode-forming part 18 with silicide-generating layer 52. Thus, portions of electrode-forming part 18 in contact with silicide-generating layer 52 may be silicided to form electrodes 15 and, as shown in FIG. 16E, in which constituent elements corresponding to those of FIG. 16C are denoted by like reference numerals, and FIG. 16F in which constituent elements corresponding to those of FIG. 16D are denoted by like reference numerals.

In some cases, at this point it may be difficult to form silicide in regions of electrode-forming part 18 underneath mask layer 19 where the silicide-generating layer 52 is not formed as a film, as shown in FIG. 16E. Silicide-generating layer 52 metal element(s) diffuses from both lateral sides of the mask layer 19 toward the regions underneath mask layer 19; siliciding also progresses in the lower regions near both lateral portions of the mask layer 19 not in direct contact with silicide-generating layer 52. Thus, electrodes 15 and 16 may be formed underneath mask layer 19 from both lateral sides of the mask layer 19. In this case, electrodes 15 and 16 may be formed in underneath mask layer 19 as the result of silicide-generating layer 52 metal element(s) diffusing from the vicinity of both lateral portions of mask layer 19, underneath mask layer 19, and thereby forming silicide. As a result, electrodes 15 and 16 expand (volumetric expansion) to a volume greater than the volume of a region of electrode-forming part 18 which mask layer does not cover. Accordingly, sidewalls 15a and 16a of electrodes 15 and 16 (specifically, curved surfaces 15c and 16c) may be formed so as to be closer to each other than the width W2 of the lower portion of mask layer 19.

Also in this case, the siliciding of electrode-forming part 18 may progress until silicon oxide layer 4 is reached. Thus, it is possible to form electrodes 15 and 16 in contact with silicon oxide layer 4. For electrodes 15 and 16 as described above, the positions of the sidewalls 15a and 16a of the electrodes 15 and 16 (curved surfaces 15c and 16c) underneath mask layer 19 can be controlled by appropriately selecting the film thickness of electrode-forming part 18, the film thickness of silicide-generating layer 52, and temperature, heating time and the like at the time of heat treatment. The minimum width W1 between sidewalls 15a and 16a can therefore be set to, for example, 0.1 nm to 30 nm, or any width as described herein, and the degree of curvature of curved surfaces 15c and 16c can be controlled.

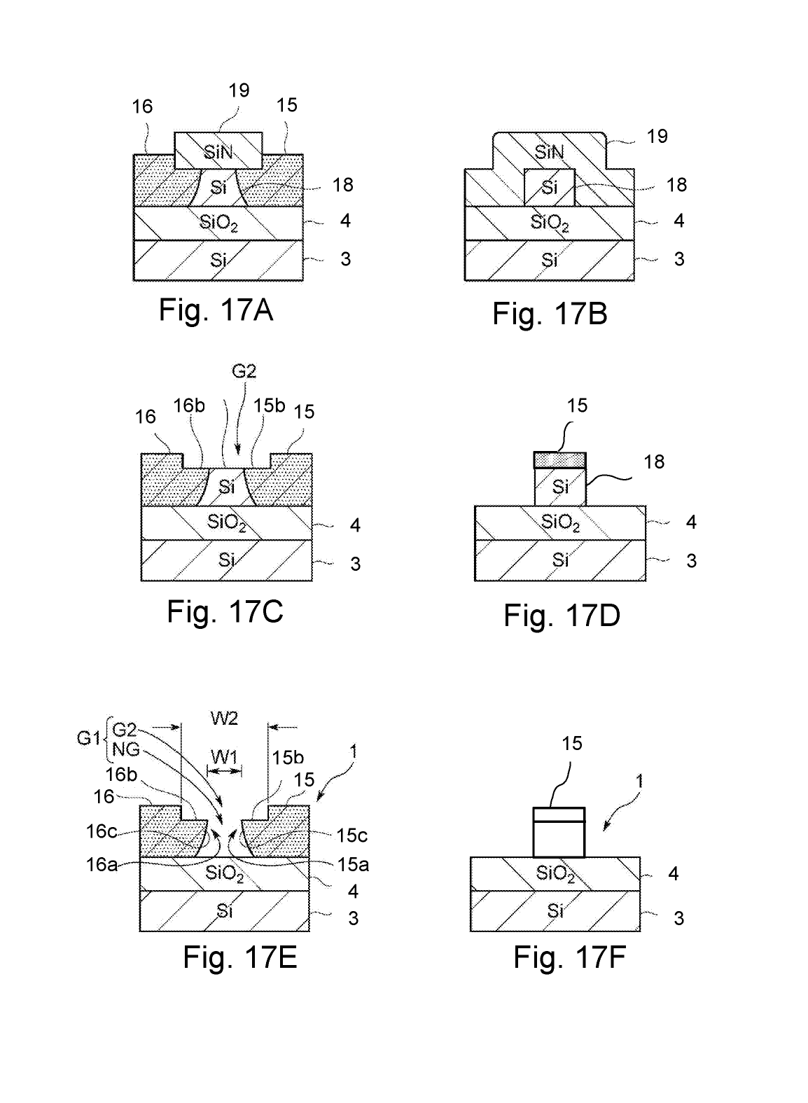

Subsequently, as shown in FIG. 17A in which constituent elements corresponding to those of FIG. 16E are denoted by like reference numerals, and FIG. 17B in which constituent elements corresponding to those of FIG. 16F are denoted by like reference numerals, unreacted portions of silicide-generating layer 52 remaining on mask layer 19 and silicon oxide layer 4 may be removed by etching. Thereafter, as shown in FIG. 17C in which constituent elements corresponding to those of FIG. 17A are denoted by like reference numerals, and FIG. 17D in which constituent elements corresponding to those of FIG. 17B are denoted by like reference numerals, mask layer 19 may be removed by etching to form mask width gap G2 between shoulders 15b and 16b of electrode parts 15 and 16.

If silicide-generating layer 52 is formed from, for example, cobalt, electrodes 15 and 16 may comprise cobalt silicide (CoSi). Thereafter, any unreacted portions of silicide-generating layer 52 remaining on mask layer 19 and silicon oxide layer 4 may be removed by wet etching using a liquid mixture of sulfuric acid (H2SO4) and hydrogen peroxide (H2O2).

In some embodiments as shown in FIG. 17E in which constituent elements corresponding to those of FIG. 17C are denoted by like reference numerals, and FIG. 17F in which constituent elements corresponding to those of FIG. 17D are denoted by like reference numerals, any unreacted portions of electrode-forming part 18 remaining between electrodes 15 and 16 on silicon oxide layer 4 may be removed by etching or the like to expose curved surfaces 15c and 16c of electrodes 15 and 16, thereby forming a hollow nano-gap NG between curved surfaces 15c and 16c. Thus, it is possible to manufacture a nano-gap electrode 1 as shown in FIG. 14.

In the above-described configuration, mask layer 19 may be selected in conformity with forming specific width, and may be formed on electrode-forming part 18, which may be located on substrate 2, and silicide-generating layer 52 may be formed as a film on electrode-forming part 18. Thereafter, a heat treatment may be performed to react silicide-generating layer 52 with electrode-forming part 18 to form two opposed electrodes 15 and 16 penetrating underneath mask layer 19 by volumetric expansion resulting from the reaction, thereby bringing sidewalls 15a and 16a of electrodes 15 and 16 closer to each other than the width of mask layer 19 by volumetric expansion. Then mask layer 19 and any unreacted portions of the electrode-forming part 18 remaining in the lower region of the mask layer 19 may be removed. A nano-gap NG can thus be formed between electrodes 15 and 16. Consequently, it is possible to manufacture a nano-gap electrode 1 having a nano-gap NG that is even smaller than mask width gap G2 formed using patterned mask layer 19.

In such a nano-gap electrode 1 as described above, the degree of penetration of the electrodes 15 and 16 from both lateral portions of the mask layer 19 underneath mask layer 19 may be controlled simply by selecting, as appropriate, a film thickness of electrode-forming part 18, a film thickness of silicide-generating layer 52, and a heat treatment time and heating temperature used to silicide electrode-forming part 18 in the course of manufacture. Thus, it is possible to easily form a nano-gap NG that is even narrower than the mask width gap G2 of mask layer 19. In addition, in such a manufacturing method as described above, it is possible to form, between electrodes 15 and 16, a nano-gap NG narrower than a mask width gap G2 having a minimum width smaller than the minimum that can be formed using lithographic techniques when mask layer 19 is used.

In some methods for manufacturing a nano-gap electrode, a nano-gap may be formed between two opposed electrodes by directly etching an electrode layer using a resist mask patterned using exposure and development. Since a minimum width that can be formed in the resist mask by exposure and development may be on the order of 10 nm, it is difficult to form a nano-gap narrower than this width using such methods.

On the other hand, in some embodiments of the methods for manufacturing a nano-gap electrode described herein, sidewalls 15a and 16a of electrodes 15 and 16 come closer to each other in the region underneath mask layer 19 due to volumetric expansion in a subsequent manufacturing process even if the minimum width W2 that can be formed in a resist mask by conventional manufacturing lithographic techniques may be 10 nm, and as a consequence, the minimum width W2 of mask layer 19 may be 5 nm to 10 nm. It is therefore possible to form a nano-gap NG having a width no greater than 2 nm, 1 nm, 0.9 nm, 0.8 nm, 0.7 nm, 0.6 nm, or 0.5 nm, or any gap spacing as described herein, which may be smaller than the minimum width W2 of 5 nm to 10 nm.

In some cases, a silicide-generating layer 52 may be formed as a film on electrode-forming part 18, and then a heat treatment may be performed; electrode-forming part 18 and silicide-generating layer 52 may thus be reacted with each other; two opposed volumetrically expanded electrodes 15 and 16 may be formed; and sidewalls 15a and 16a of electrodes 15 and 16 may be brought closer to each other by volumetric expansion, thereby forming nano-gap NG between electrodes 15 and 16. It is therefore possible to make mask width gap G2 between electrodes 15 and 16 smaller by as much as the amount of silicidation. Consequently, it is possible to manufacture a nano-gap electrode 1 having a nano-gap NG that is even smaller than a gap formed by conventional lithographic processing.

In such a manufacturing method as described above, it is possible to form curved surfaces 15c and 16c whereby opposed sidewalls 15a and 16a of electrodes 15 and 16 may be gradually brought closer to each other. It is therefore possible to manufacture a nano-gap electrode 1 in which the width between sidewalls 15a and 16a gradually narrows due to the curvature of curved surfaces 15c and 16c.

In some cases, electrodes 15 and 16 may be formed so as to be in contact with silicon oxide layer 4. As an alternative, electrodes 15 and 16 need not be formed so as to be in contact with silicon oxide layer 4, and an unreacted portion of electrode-forming part 18 may be formed between silicon oxide layer 4 and electrodes 15 and 16. In this embodiment, it is possible for the unreacted portion of electrode-forming part 18 to remain between silicon oxide layer 4 and electrodes 15 and 16 by appropriately selecting a film thickness for electrode-forming part 18 and silicide-generating layer 52 and a heat treatment time and temperature for siliciding (or silicidation) electrode-forming part 18.

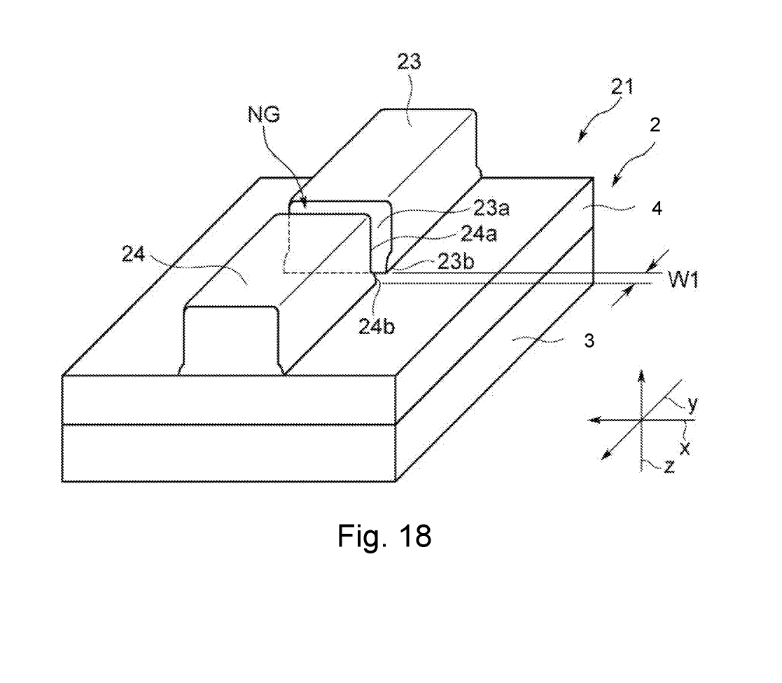

In another embodiment as illustrated in FIG. 18, in which constituent elements corresponding to those of FIG. 14 are denoted by like reference numerals, a nano-gap electrode 21 is shown. A nano-gap electrode 21 is depicted which has a nano-gap NG with a minimum width W1, which is nanoscale (no greater than 1000 nm), may be formed between electrodes 23 and 24. Nano-gap electrode 21 is characterized in that it is possible to form nano-gap NG narrower than the width of a mask width gap formed using a mask using standard lithographic processes. Nano-gap NG may be formed with a minimum width W1 of 0.1 nm to 30 nm, or no greater than 2 nm, 1 nm, 0.9 nm, 0.8 nm, 0.7 nm, 0.6 nm, or 0.5 nm, or may be of any width as described herein.

Electrodes 23 and 24 may be formed from one or more of various types of metal silicide, including titanium silicide, molybdenum silicide, platinum silicide, nickel silicide, cobalt silicide, palladium silicide, and niobium silicide, or combinations thereof. Electrodes 23 and 24 may have the same configuration and may be formed bilaterally symmetrically across nano-gap NG on substrate 2. Sidewalls 23a and 24a at respective ends of electrodes 23 and 24 may be disposed opposite to each other across nano-gap NG. In some embodiments, electrodes 23 and 24 may comprise rectangular solids, the longitudinal cross section of which may be quadrilateral, and the longitudinal axis of which may extend in a y-direction. Electrodes 23 and 24 may be disposed so that the long-side central axes thereof may be positioned on the same y-axis straight line and may be positioned such that the front surfaces of sidewalls 23a and 24a may face each other.

In some embodiments, outward-expanding portions may be formed in the regions of the sidewalls 23a and 24a of electrodes 23 and 24 in contact with substrate 2. Consequently, electrodes 23 and 24 allow the width of nano-gap NG formed therebetween to be further narrowed to a minimum width W1 in a region in which expanded portions 23b and 24b face each other.

In some embodiments, utilizing nano-gap electrode 21, current can be supplied from, for example, a power source (not shown) to the electrodes 23 and 24, and the value of a current between electrodes 23 and 24 may be measured with an ammeter (not shown). Accordingly, nano-gap electrode 21 allows single-stranded DNA to pass through nano-gap NG between electrodes 23 and 24 from an x-axis orthogonal to the y-axis, which may be the longitudinal axis of electrodes 23 and 24, and/or from a z-axis, which may be the height axis of electrodes 23 and 24 and intersects at right angles with the y-axis; an ammeter may be used to measure the values of currents flowing across electrodes 23 and 24 when bases of the single-stranded DNA pass through nano-gap NG between electrodes 23 and 24; and the bases comprising single-stranded DNA may be determined on the basis of the current values.

In some embodiments a method for manufacturing may be utilized for fabricating a nano-gap electrode 21 comprising a substrate 2 wherein a silicon oxide layer 4 may be formed on a silicon substrate 3 may be prepared, and a silicon layer may thence be formed on silicon oxide layer 4. Subsequently, a resist layer may be formed as a film on this silicon layer, and this resist layer may then be patterned by exposure and development to form a mask (resist mask).

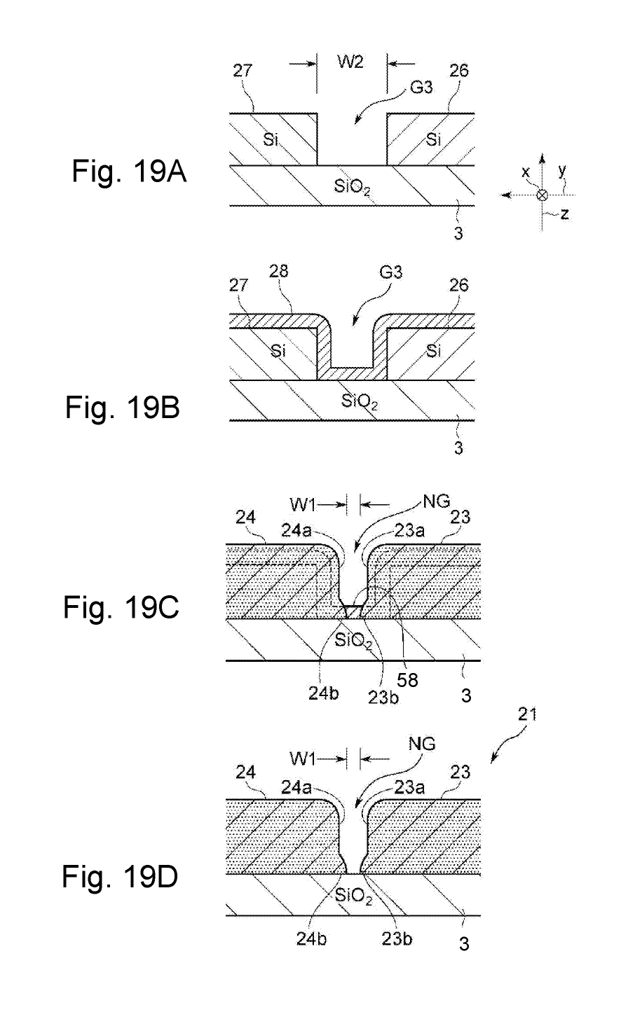

Subsequently, the silicon layer may be patterned using the mask. Then, as shown in FIG. 19A, two electrode-forming parts 56 and 57 which may be opposed to each other across mask width gap G3 may be formed from the silicon layer. Note that in this case, electrode-forming parts 56 and 57 may be formed into a solid shape, which may be rectangular, which may have a longitudinal axis direction extending parallel the y-axis. In addition, electrode-forming parts 56 and 57 may be disposed so that the long-side central axes thereof may be positioned on the same straight line and so that sidewalls of electrode-forming parts 56 and 57 may face each other across mask width gap G3.

In some embodiments as shown in FIG. 19B in which constituent elements corresponding to those of FIG. 19A are denoted by like reference numerals, a silicide-generating layer 58 may be made from a metal element, such as titanium, molybdenum, platinum, nickel, cobalt, palladium or niobium or combinations or alloys thereof, may be formed as a film on electrode-forming parts 56 and 57 and on an exposed portion of silicon oxide layer 4 by, for example, sputtering. Subsequently, a heat treatment may be performed to react electrode-forming parts 56 and 57 with silicide-generating layer 58. Thus, electrode-forming parts 56 and 57 which may be in contact with silicide-generating layer 58 may form a silicide, producing electrodes 23 and 24 made from metal silicide, as shown in FIG. 19C in which constituent elements corresponding to those of FIG. 19B are denoted by like reference numerals.

Here, electrodes 23 and 24, when made silicide, volumetrically expand, and therefore sidewalls 23a and 24a come closer to each other. Thus, it is possible to form a nano-gap NG much narrower than mask width gap G3 formed using the mask. At this time, any excess amounts of silicide-generating layer 58 may be present in regions of the electrode-forming parts 56 and 57 in contact with the substrate 2, compared with other regions. Hence, siliciding of electrode-forming parts 56 and 57 in conjunction with the silicide-generating layer 58 may be facilitated in those regions. Formation of electrodes 23 and 24 may cause further volumetric expansion resulting in expanded portions 23b and 24b. Consequently, the electrodes 23 and 24 can be formed so that the width of nano-gap NG may be further narrowed by the formation of expanded portions 23b and 24b disposed opposite to each other in the regions where electrodes 23 and 24 contact substrate 2.

For electrodes 23 and 24 which are formed using this method, the positions of sidewalls 23a and 24a of electrodes 23 and 24 and the degree of expansion of the expanded portions 23b and 24b may be controlled by appropriately selecting the film thicknesses of electrode-forming parts 56 and 57, the film thickness of silicide-generating layer 58, and the temperature, heating time and the like at the time of heat treatment. The width between sidewalls 23a and 24a and the minimum width W1 between expanded portions 23b and 24b can therefore be set to, for example, from 0.1 nm to 30 nm, or no greater than 2 nm, 1 nm, 0.9 nm, 0.8 nm, 0.7 nm, 0.6 nm, or 0.5 nm, or any gap spacing as described herein.

Subsequently, any unreacted portions of the silicide-generating layer 58 remaining on the silicon oxide layer 4 within the nano-gap NG and in other regions may be removed by etching, as shown in FIG. 19D in which constituent elements corresponding to those of FIG. 19C are denoted by like reference numerals. Thus, it is possible to manufacture nano-gap electrode 21 having nano-gap NG between electrodes 23 and 24, as shown in FIG. 18.

In the above-described configuration, the two electrode-forming parts 56 and 57 disposed opposite to each other across the gap (mask width gap G3) may be formed on substrate 2; silicide-generating layer 58 may be formed as a film on electrode-forming parts 56 and 57; and then a heat treatment may be performed to react silicide-generating layer 58 with electrode-forming parts 56 and 57, thereby forming two opposed electrodes 23 and 24 which may be volumetrically expanded due to the reaction. Thus, it is possible to bring the sidewalls 23a and 24a of electrodes 23 and 24 closer to each other by volumetric expansion and to form nano-gap NG smaller than mask width gap G3 formed between electrodes 23 and 24 which can normally be fabricated using lithographic methods. Consequently, it is possible to manufacture the nano-gap electrode 21 having a nano-gap NG even smaller than mask width gap G3 formed using the patterned mask.

In some embodiments when forming a nano-gap electrode 21 as described above, the degree of volumetric expansion of electrodes 23 and 24 may be controlled simply by selecting, as appropriate, the film thicknesses of electrode-forming parts 56 and 57, a film thickness of silicide-generating layer 58, and heat treatment time and heating temperature used to silicide electrode-forming parts 56 and 57 in the course of manufacture. Thus, it is possible to form a nano-gap NG even narrower than mask width gap G3 of associated with a mask. In some cases, between electrodes 23 and 24 may be formed a nano-gap NG narrower than a mask width gap G3 having the minimum width that can be formed with the mask using standard lithographic processes.

In some embodiments, the silicide-generating layer 58 may be formed as a film on electrode-forming parts 56 and 57, and then a heat treatment may be performed; electrode-forming parts 56 and 57 and silicide-generating layer 58 may thus be reacted with each other; two opposed volumetrically expanded electrodes 23 and 24 may be formed; and sidewalls 23a and 24a of electrodes 23 and 24 may be brought closer to each other by volumetric expansion, thereby forming a nano-gap NG between electrodes 23 and 24. It is therefore possible to make mask width gap G3 between electrodes 23 and 24 smaller by as much as the amount of volumetric expansion. Consequently, it is possible to manufacture nano-gap electrode 21 having a nano-gap NG even smaller than a gap formed by normal (or standard) lithographic processing.

In some embodiments, it is possible to form expanded portions 23b and 24b whereby opposed sidewalls 23a and 24a of electrodes 23 and 24 may be gradually brought closer to each other. It is therefore possible to manufacture a nano-gap electrode 21 in which the width between sidewalls 23a and 24a gradually narrows due to the growth of expanded portions 23b and 24b.

It will be apparent to those skilled in the art that the present invention is not limited to the present embodiments, and it may be modified and carried out in various other ways within the scope of the subject matter of the present invention. For example, the electrodes 15 and 16 (23 and 24) may have various shapes. In some cases, electrode-forming part(s) 18 (26 and 57) may be made from silicon, the silicide-generating layer 52 (28) may be made from one or more metal elements, such as titanium, molybdenum, platinum, nickel, cobalt, palladium or niobium or alloys thereof, which may be formed as a film on electrode-forming part(s) 18 (56 and 57). A heat treatment may then be performed to react electrode-forming part(s) 18 (56 and 57) with silicide-generating layer 52 (28), thereby forming volumetrically expanded electrodes 15 and 16 (23 and 24) made from metal silicide(s). The present invention is not limited to these embodiments, however. Alternatively, an electrode-forming part made from titanium may be formed; a compound-generating layer made from tungsten may be formed as a film on the electrode-forming part; a heat treatment may be performed thereafter to react the electrode-forming part with the compound-generating layer; and volumetrically expanded electrodes made from titanium tungsten may be formed, thereby forming a nano-gap between the electrodes with the sidewalls of electrodes brought closer to each other by as much as the amount of volumetric expansion. It will be appreciated that materials other than titanium and tungsten may be used.

Also in the above-described first and second embodiments, a description has been made of a nano-gap electrode 1 (21) in which single-stranded DNA may be passed through a nano-gap NG between electrodes 15 and 16 (23 and 24), and the values of current flowing across or between electrodes 15 and 16 (23 and 24) when bases of single-stranded DNA pass through nano-gap NG between electrodes 15 and 16 (23 and 24) and may be measured with an ammeter. The present invention is not limited to these embodiments, however. The nano-gap electrode may be used in various other applications.

In some embodiments a method for manufacturing may be utilized for fabricating a nano-gap electrode 21 comprising a substrate 2 wherein a silicon oxide layer 4 may be formed on which a silicon substrate 3 may be prepared, and a silicon layer may thence be formed on silicon oxide layer 4. Subsequently, a resist layer may be formed as a film on this silicon layer, and this resist layer may then be patterned by exposure and development to form a mask (resist mask).

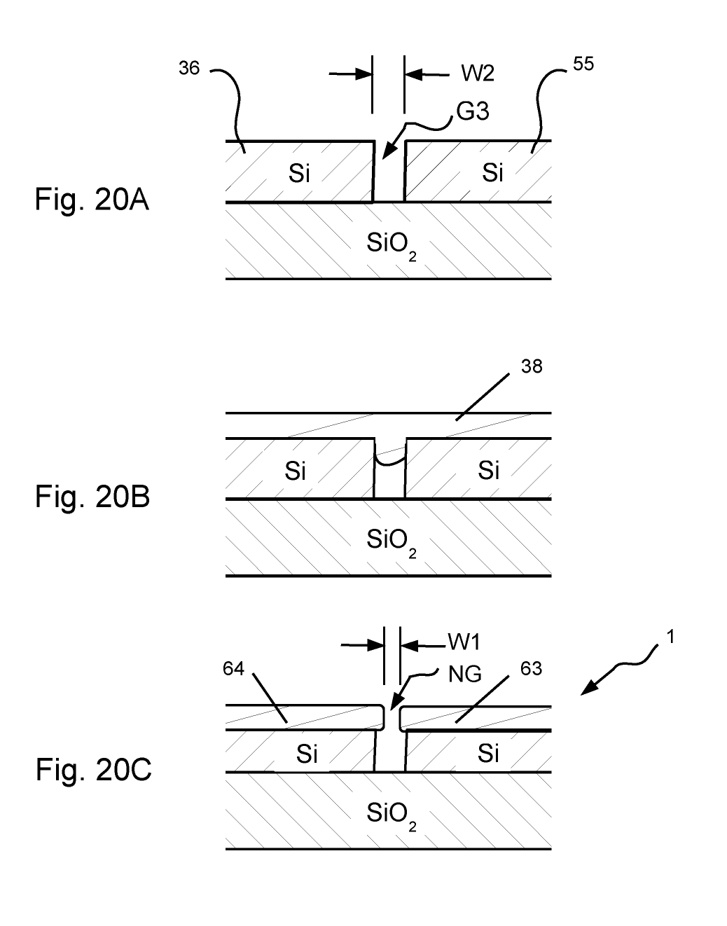

Subsequently, the silicon layer may be patterned using the mask. Then, as shown in FIG. 20A, two electrode-forming parts 55 and 36 which may be opposed to each other across mask width gap G3 may be formed from the silicon layer. Note that in this case, electrode-forming parts 55 and 36 may be formed into a solid shape, which may be rectangular, and which may have a longitudinal axis direction extending parallel to the y-axis. In addition, electrode-forming parts 55 and 36 may be disposed so that the long-side central axes thereof may be positioned on the same straight line and so that sidewalls of electrode-forming parts 55 and 36 may face each other across mask width gap G3.

Subsequently, as shown in FIG. 20B in which constituent elements corresponding to those of FIG. 20A are denoted by like reference numerals, a silicide-generating layer 38 may be made from a metal element, such as titanium, molybdenum, platinum, nickel, cobalt, palladium, niobium, or any other transitional metal or combinations or alloys thereof, may be formed as a film on electrode-forming parts 55 and 36 by, for example, sputtering. In some embodiments the sputtering may be done at an angle. Due to the narrowness of mask width gap G3 silicide-generating layer 38 may not reach the bottom.

Subsequently, a heat treatment may be performed to react electrode-forming parts 55 and 36 with silicide-generating layer 38, which may be in a salicide or polycide process. Subsequently, any unreacted portions of the silicide-generating layer 38 remaining above silicon oxide layer 4 within nano-gap NG and in other regions may be removed by etching. Thus, electrode-forming parts 55 and 36, which may be in contact with silicide-generating layer 38, may form silicided electrodes 63 and 64, made from metal silicide, as shown in FIG. 20C in which constituent elements corresponding to those of FIG. 20B are denoted by like reference numerals.

Thus side walls of electrodes 63 and 64 may be brought closer to each other by volumetric expansion, thereby forming nano-gap NG between electrodes 63 and 64. It is therefore possible to make mask width gap G3 between electrodes 23 and 24 smaller by as much as the amount of volumetric expansion. Consequently, it is possible to manufacture nano-gap electrode 1 having a nano-gap NG even smaller than a gap formed by normal lithographic processing.

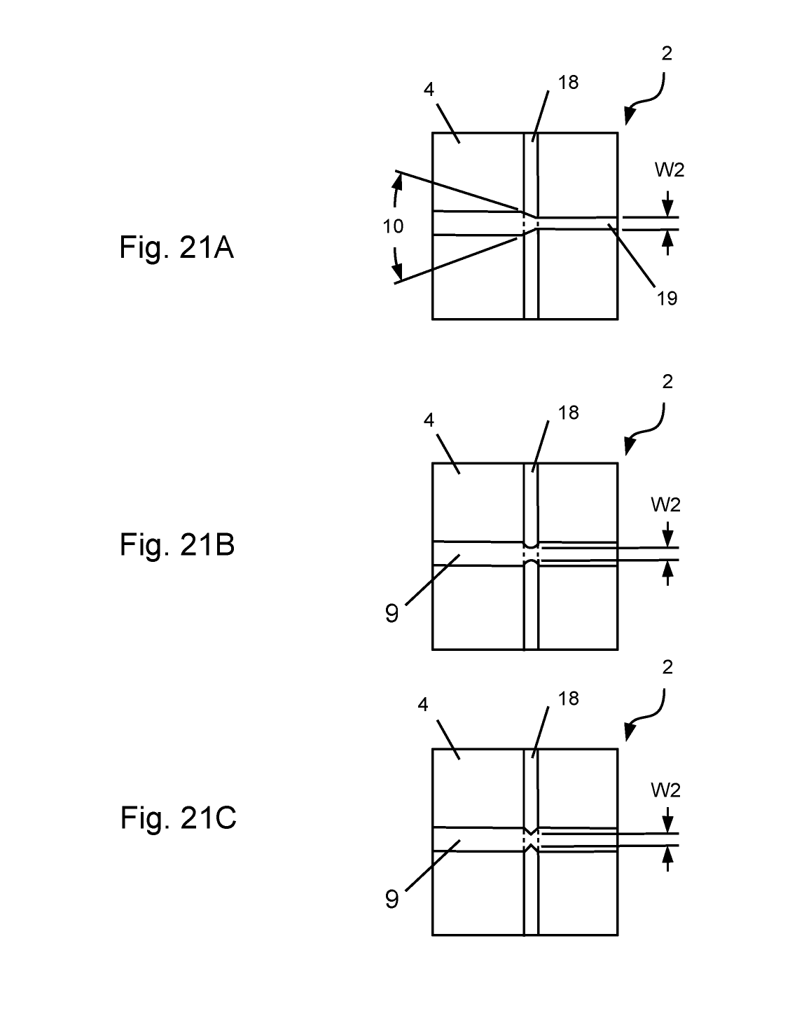

In some embodiments it may be desirable to use a non-rectangularly shaped mask layer 19. This can advantageously create a point or vertical edge for nano-gap NG to better facilitate single base measurements. FIGS. 21A-21C show top views of three different mask variations where the minimum mask dimension may be the width W2 corresponding to mask width gap G2. In one embodiment as shown in FIG. 21A the mask creates a trapezoidally shaped gap film on an electrode-forming part 18. In some embodiments the trapezoidal angle 10 may be greater than or equal to 10 degrees, greater than or equal to 30 degrees, or greater than or equal to 60 degrees. In some embodiments the silicide formed by diffusion of metal into silicon will result in electrodes having curved rather than planar edges, but may still have a minimum gap distance G2. The present invention is not limited to the masks variations shown in FIGS. 21A-21C.

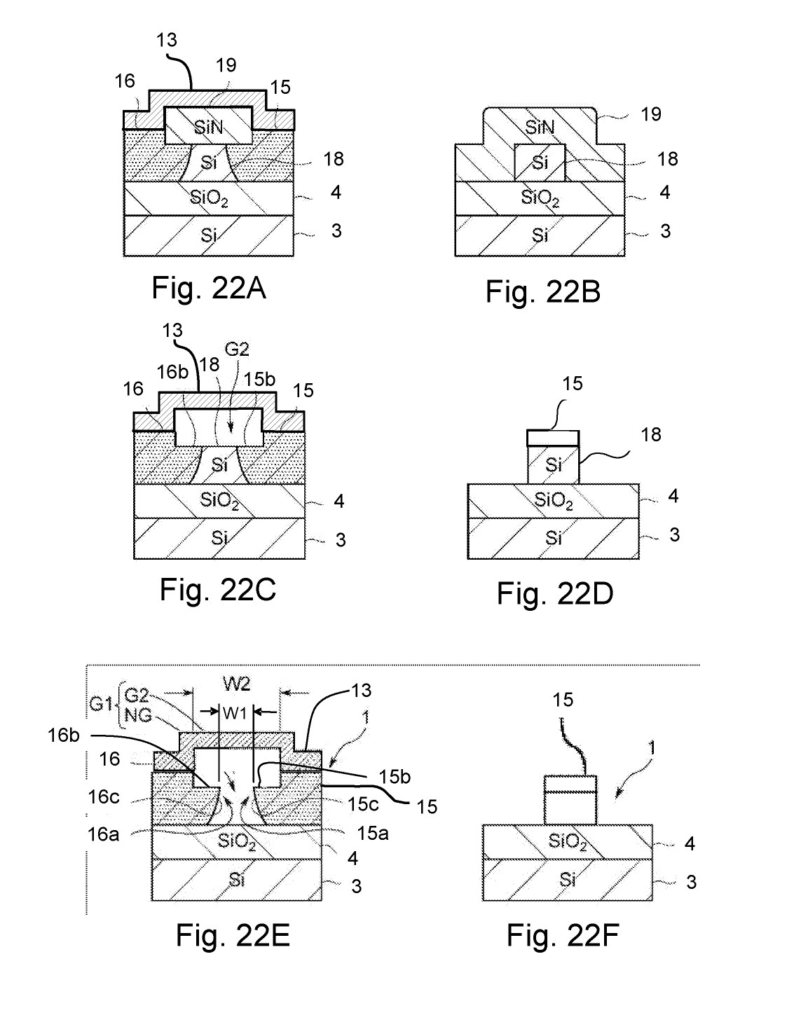

In some embodiments as shown in FIGS. 22A-22F in which constituent elements corresponding to those of FIGS. 20A-20F are denoted by like reference numerals it may be desirable to form small channels to bring a target species (e.g., a biomolecule such as DNA or RNA) to the nanogap electrodes. Mask layer 19 may be designed to form this channel, as it may be etched away during the process. FIGS. 22A, 22C and 22E show the addition of a channel top layer 13. The channel top layer 13 is not shown in 22B, 22D and 22E for clarity. In some embodiments the channel top layer may be a nonconducting material compatible with the fabrication methods such as SiO₂ or may be a polymer such as polydimethylsiloxane or SU8.

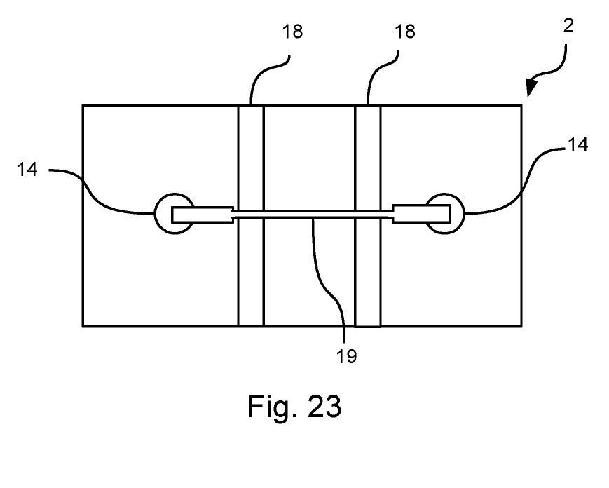

In some embodiments as shown in FIG. 23, in order to enable etching away of the mask layer 19 the channel top layer 13 may be deposited with at least one channel access port 14. In FIG. 23 a top view is shown with two channel access ports 14. In some embodiments the width and thickness of the mask layer 19 may be varied along the axis of the mask axis, which when removed may form one or more channels. In some embodiments multiple electrode pairs may be situated in each channel.

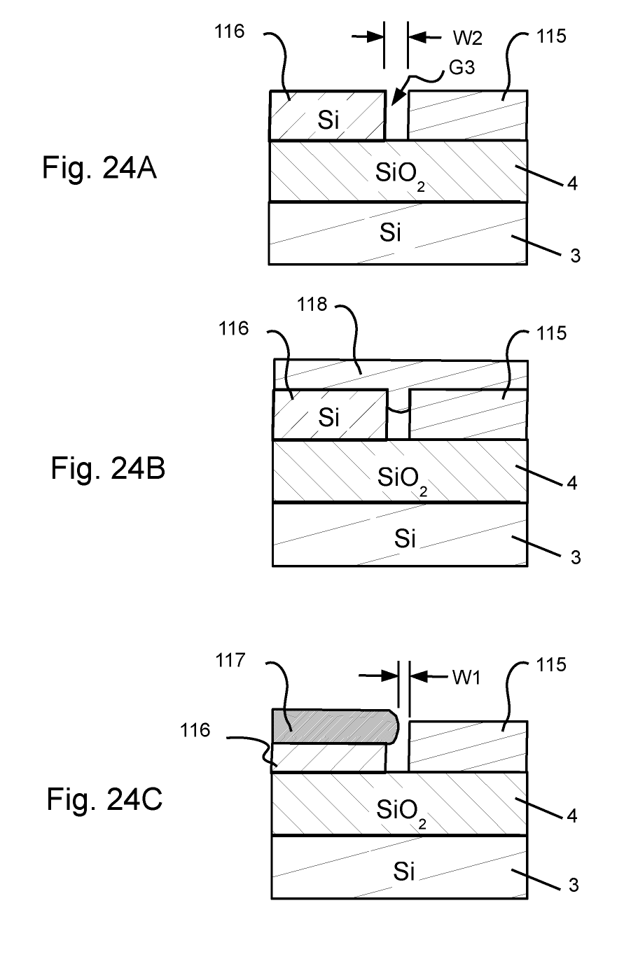

In some embodiments as shown in FIGS. 24A-24B the silicide expansion may be done from only one side. In some embodiments electrode forming part 116 and metal electrode 115 may be fabricated. Subsequently silicide-generating layer 118 may be formed as a film using, for example sputtering. As shown in FIG. 24A the gap W2 may be sufficiently narrow such that silicide-generating layer 118 may not extend all the way down the bottom of gap W2. The metal of the metal electrode 115 may be selected with respect to the silicide-generating layer 118 such that the silicide-generating layer 118 may be etched away without affecting the metal electrode 115.

Subsequently, a heat treatment may be performed to react electrode-forming parts 116 with silicide-generating layer 118 to form electrode 117. Any unreacted portions of silicide-generating layer 118 remaining on the silicon oxide layer 4 within the nano-gap NG and in other regions may be removed by etching. As shown in FIG. 24B the expansion of the silicide can create a gap of width W1 that is narrower than the mask width W2.

In some embodiments resulting silicide(s) may be conductive. The silicide(s) formed may be formed in a self-aligned process such as a salicide process or a polycide process. Multiple silicide generating processes may be utilized for the same electrode forming elements, for example, to form electrodes and electrode tips, and to connect to interconnects whereby currents, which may pass through the electrodes tips, and may thence pass to an amplifier or measurement device. Interconnects may also be utilized to apply a bias potential, which may originate from a bias source, be carried by interconnect(s) and applied to electrode(s) which may be formed of a silicide material which may have been formed using a salicide process.

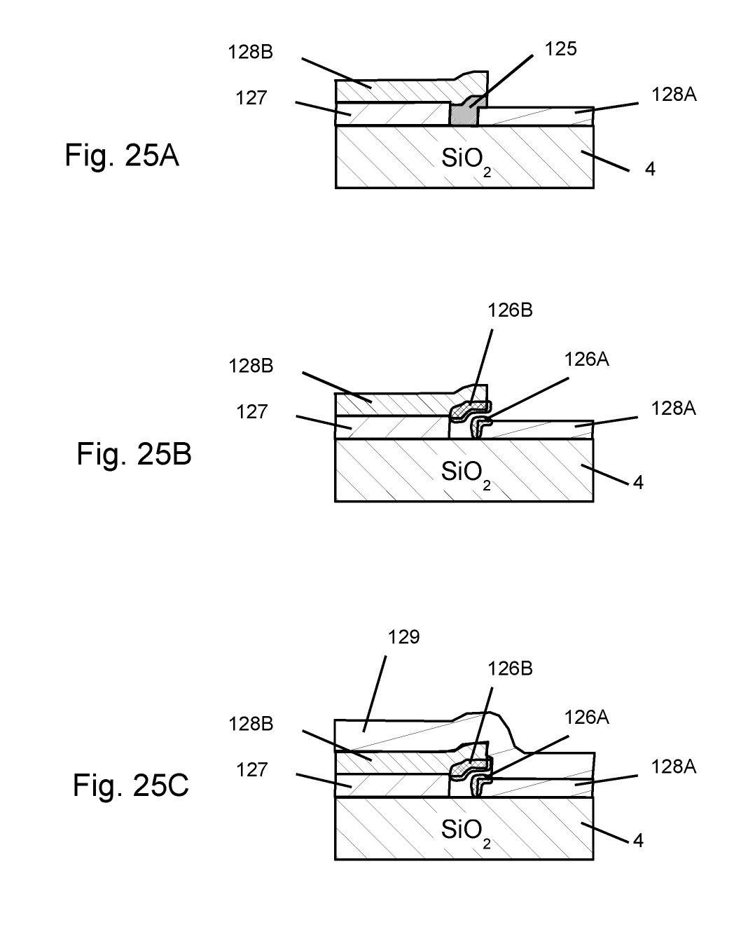

In some embodiments the silicide expansion can create a vertical nano-gap. An electrode forming part 125 and a first silicide-generating electrode 128a may be fabricated first on a SiO2 coated wafer as shown in FIG. 25A. This may be followed by a dielectric layer 127, such as SiO2. Subsequently a second silicide-generating electrode 128b may be deposited. This is shown in FIG. 25B.

Subsequently, as shown in FIG. 25C a heat treatment may be performed to react electrode-forming part 125 with silicide-generating layers 128a and 128b. The non-reacted portion of the electrode forming part 125 may be then etched away. This may be followed by a dielectric cover 129 with one or more axis holes (not shown) to provide fluidic channel created by the removal of the residual of the electrode forming part 125. The completed cross section is shown in FIG. 25D.

In some cases, mask width gaps G2 and G3, which may be, formed using a patterned mask, may be applied as gaps previously formed by processing when nano-gap NG is formed. The present invention is not limited to these embodiments, however. In the one embodiment, a gap may be formed by first forming mask width gap G2 using patterned mask layer 19, and then further trimming the pattern of the mask to control the gap of mask layer 19. In another embodiment, a gap may be formed by, for example, narrowing the gap between electrode-forming parts 56 and 57 by deposition, or by various other types of processes. In the present invention, a gap can be made smaller by as much as the amount of volumetric expansion of electrode parts, as described above. Consequently, it is possible to manufacture a nano-gap electrode having a nano-gap NG that is even smaller than a gap formed by normal lithographic processing.

In some embodiments, a nanochannel may be made to be smaller, wherein smaller may be a decrease in the width of the channel or the depth of the channel, or may be a decrease of both the width and the depth of the channel. In some embodiments, techniques as described herein may be utilized to narrow one or both of the width and depth of a channel.

In some embodiments, the width and/or depth of a channel may be decreased using the same or similar process as that used to form the nano-gap. In some cases, alternative or additional process operations may be utilized to decrease the width and/or depth of a channel. In some embodiments, wherein a material utilized to decrease the width and/or depth of a channel may be considered to be non-conducting, the material may be let exposed, and may form the wall of a channel.

In other embodiments, wherein a material utilized to decrease the width and/or depth of a channel may be considered to be a conductor, a non-conducting material may be overlaid over the conducting material, so as to prevent interference with normal use of the channel, which may include the use of electrophoretic translocation of biomolecules through a channel. A material which may be utilized as a nonconductor covering a conductive material utilized to narrow a channel may comprise SiO₂, or other oxides typically utilized in semiconductor processes.

In other embodiments wherein a material which may be considered to be a conductor may be utilized to decrease the width and/or depth of a channel, different portions of the channel may be left without the material utilized to reduce the width of the channel, thereby segmenting the conducting material, which may thereby prevent interference with a use of electrophoresis for translocation.

In other embodiments, a material utilized to reduce the width and/or depth of a channel may be utilized in some sections of a channel and not in others. For example, a material utilized to reduce the width and/or depth of a channel may be utilized to reduce the width and/or depth of channel in the immediate vicinity of a nano-gap electrode, so as to increase the probability of interaction between a biomolecule which may be being translocated through a channel and a nano-gap electrode which may be positioned so as to interrogate molecules translocating through a channel. A material utilized to reduce the width and/or depth of a channel may be utilized so as to reduce the width and/or depth of a channel at a distance close enough to a nano-gap so as to prevent formation of secondary structure adjacent to a nano-gap electrode.

In some embodiments, a material used to reduce the width and/or depth of a channel may immediately juxtapose materials used to form a nano-gap electrode, particularly if the material utilized to reduce the width and/or depth of a nano channel is a non-conductor. In other embodiments, wherein a material utilized to reduce the width and/or depth of a nano-gap may be considered to be a conductor, a spacer element may be desired between an electrode structure and the material utilized to narrow a width and/or depth of a channel.

A spacer element used to space an electrode and a conductive material utilized to narrow a width and/or depth of a channel may comprise a nonconductive material, which may at least be partly be left in place during the use of a channel structure, or may comprise a conductive or nonconductive material which may be removed after the decreasing of the width and/or depth of a channel.

In some embodiments, both sides of a channel may be narrowed, while in other embodiments, a single side of a channel may be narrowed.

In some embodiments, such as shown in FIG. 3E, a sidewall 11 may be formed and layers of TiN which form electrodes 5 and 6 may be etched back exposing both sides of sidewall 11, sidewall may be widened using any of the techniques described herein, and a nonconductor may be applied, which may fill in the space between the widened sidewall 11 electrodes 5 and 6, and nanochannel walls (not shown). A non-conductor may comprise SiO₂, which may be applied using any standard semiconductor process such as CVD which may comprise low pressure CVD (LPCVD) or ultra-low vacuum CVD (ULVCVD), plasma methods such as microwave enhanced CVD or plasma enhanced CVD, atomic layer CVD, atomic layer deposition (ALD) or plasma-enhanced ALD, vapor phase epitaxy, or any other appropriate fabrication method. The structure may be polished (e.g., using CMP) and over polished so as to set a desired depth for a channel.

In other embodiments as shown in FIG. 8A, side walls 37 may be formed with a width that corresponds to a minimum semiconductor fabrication feature dimension; a mask layer which may be a resist mask may be placed over sidewall forming mask 40, side wall 37, electrode supporting part 29, and electrode forming part 31. An additional layer may be added to sidewall 37, thereby increasing the thickness which corresponds to the width of the channel thereby.

In some embodiments similar to those shown in FIGS. 17A-F which depict the fabrication of a narrow nano-gap, expanded electrode parts 15 and 16 may be prevented from coming in contact with a channel narrowing material by utilizing a material in a manner similar to that of an electrode forming part 18, which may extend the length of the channel, with a gap between the electrode portion and the section of channel immediately adjacent, wherein in silicidation of the electrode forming part and the similar material used to narrow a channel may thus be caused to narrow the electrode gap and channel respectively. Mask layer 19 may be deposited in the gap between a channel and an electrode structure providing an electrically isolating barrier between two conductive materials, preventing shorting of different electrodes which may be placed at various positions along a channel.

In some embodiments mask layer 19 may be utilized to increase the width of a channel by increasing the width of mask layer 19, such that subsequent formation of silicides thereunder will start from positions further apart, and will therefore result in spacings betwixt which will be accordingly larger.

In some embodiments, the width and/or depth of a channel may be consistent along its length, while in other embodiments, the width and/or depth of a channel may vary, wherein the width and/or depth of a channel may be narrower in the vicinity of an electrode structure, and may widen elsewhere. For embodiments wherein multiple electrode structures are positioned along a single nano channel, the width and/or depth of a channel may be matched to the spacing of the electrode gap in the vicinity of electrode structures, and may widen between electrode structures.

In some embodiments wherein the spacing of electrodes may be narrower than the diameter of a target molecule, which may be a biomolecule (e.g., DNA or RNA), in matching the spacing of an electrode gap, a channel may be larger than the width of an electrode gap. In some cases, the channel is from 0.1 nm wider than an electrode gap to 0.3 nm wider than an electrode gap, or from 0.1 nm to 1 nm wider than an electrode gap, or from 0.1 nm to 3 nm wider than an electrode gap. Similarly, the depth of a channel may be larger than the width of an electrode gap when a biomolecule is larger than the spacing of an electrode gap, and may be dimensioned similarly to the width.

In other embodiments, the width of a channel may be larger or smaller than the depth of a channel. In some embodiments, the depth of a channel may be less than the diameter of a biomolecule, where in the diameter may be considered to be the distance of, for example of half the diameter of double stranded DNA, for at least a part of a channel near a nanogap, such that a biomolecule may be constrained to be oriented such that it may be likely to interact with the electrodes of an electrode gap.

In other embodiments, wherein a channel may vary in width and/or depth, a channel may not be narrowed for portions of a channel, for example, portions of a nanochannel between electrode nano-gaps which may be spaced along a nanochannel.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for manufacturing a sensor having at least one nano-gap, comprising:
(a) providing a first electrode-forming part adjacent to a substrate, a sidewall adjacent to the first electrode-forming part, and a second electrode-forming part adjacent to the sidewall;
(b) removing the sidewall, thereby forming a nano-gap between the first electrode-forming part and the second electrode-forming part; and
(c) preparing the first electrode-forming part and the second electrode-forming part for use as electrodes that detect a current across the nano-gap when a target species is disposed therebetween.

2. The method of claim 1, wherein preparing the first electrode-forming part and the second electrode-forming part for use as the electrodes comprises removing at least a portion of the first electrode-forming part and the second electrode-forming part to provide the electrodes.

3. The method of claim 1, wherein the first and/or second electrode-forming part is formed of a metal nitride.

4. (canceled)

5. The method of claim 1, wherein the substrate comprises a semiconductor oxide layer adjacent to a semiconductor layer.

6. (canceled)

7. The method of claim 1, wherein the sidewall has a width that is less than or equal to about 2 nanometers.

8. (canceled)

9. (canceled)

10. The method of claim 1, wherein the target species is a nucleic acid molecule, and wherein the sidewall has a width that is less than a diameter of the nucleic acid molecule.

11. The method of claim 1, further comprising, prior to (c), exposing surfaces of the first electrode-forming part, the sidewall and the second electrode-forming part.

12. The method of claim 1, further comprising, prior to (b), removing a portion of the sidewall such that a cross section of the sidewall between first electrode-forming part and the second electrode-forming part has a quadrilateral shape.

13. The method of claim 1, further comprising forming a channel intersecting the nano-gap.

14. (canceled)

15. A method for forming a sensor having at least one nano-gap, comprising:
(a) disposing a gap-forming mask having lateral walls opposed to each other across a gap on an electrode-forming part that is adjacent to a substrate, wherein the gap has a first width;
(b) forming sidewalls on the lateral walls of the gap-forming mask, wherein the electrode-forming part is exposed between the sidewalls;
(c) removing a portion of the electrode-forming part exposed between the sidewalls to form a nano-gap therebetween, wherein the nano-gap has a second width that is less than the first width;
(d) removing the sidewalls to expose portions of the electrode-forming part separated by the nano-gap; and
(e) preparing the portions of the electrode-forming part for use as electrodes that detect a current across the nano-gap when a target species is disposed therebetween.

16. (canceled)

17. (canceled)

18. (canceled)

19. The method of claim 15, wherein the second width is less than or equal to about 2 nanometers.

20. (canceled)

21. (canceled)

22. (canceled)

23. (canceled)

24. (canceled)

25. (canceled)

26. A method for forming a sensor having at least one nano-gap, comprising:
(a) providing a mask comprising a sidewall, wherein the sidewall is disposed adjacent to an electrode-forming part that is adjacent to a substrate;
(b) removing the sidewall to form a gap in the mask, wherein the gap exposes a portion of the electrode-forming part;
(c) removing the portion of the electrode-forming part to form a nano-gap;
(d) removing the mask to expose portions of the electrode-forming part separated by the nano-gap; and
(e) preparing the portions of the electrode-forming part for use as electrodes that detect a current across the nano-gap when a target species is disposed therebetween.

27. (canceled)

28. The method of claim 26, wherein (a) comprises (i) providing the sidewall on a lateral wall of a first mask disposed adjacent to the electrode-forming part, (ii) removing the first mask, and (iii) forming a second mask adjacent to the sidewall, wherein the mask comprises at least a portion of the second mask.

29. (canceled)

30. (canceled)

31. (canceled)

32. The method of claim 26, wherein (a) comprises (i) providing the sidewall on a lateral wall of a first mask disposed adjacent to the electrode-forming part, (ii) forming a second mask adjacent to the sidewall, and (iii) etching the second mask, wherein the mask comprises at least a portion of the first mask and the second mask.

33. (canceled)

34. (canceled)

35. (canceled)

36. (canceled)

37. (canceled)

38. (canceled)

39. The method of claim 26, wherein (a) further comprises providing a side-wall forming layer and etching the side-wall forming layer to form the sidewall.

40. (canceled)

41. (canceled)

42. (canceled)

43. (canceled)

44. (canceled)

45. (canceled)

46. A method of manufacturing a nano-gap electrode sensor, comprising:
(a) providing a film having a first material on an electrode-forming part having a second material, wherein the electrode-forming part is disposed adjacent to a substrate;
(b) heating the film to react the first and second materials, thereby forming two electrode parts volumetrically expanded and opposed to each other, wherein each of the electrode parts has a sidewall;
(c) bringing sidewalls of the electrode parts towards each other by volumetric expansion, thereby forming a nano-gap between the electrode parts; and
(d) preparing the electrode parts for use as electrodes that detect a current across the nano-gap when a target species is disposed therebetween.

47. (canceled)

48. The method of claim 46, wherein (a) comprises (i) forming a mask selected in conformity with a width of the electrode-forming part, (ii) forming the film on the electrode-forming part.

49. (canceled)

50. (canceled)

51. (canceled)

52. (canceled)

53. A method of manufacturing a sensor having at least one nano-gap electrode, comprising:
(a) providing two electrode-forming parts adjacent to a substrate, wherein the electrode-forming parts are disposed opposite one another across a gap having a first width;
(b) forming a film of a compound-generating layer on the electrode-forming parts;
(c) performing a heat treatment to facilitate a reaction between the compound-generating layer and at least one of the electrode-forming parts to form at least one electrode part volumetrically expanded by the reaction, thereby bringing sidewalls of the electrode-forming parts towards each other by volumetric expansion to form a nano-gap having a second width smaller than the first width; and
(d) preparing the electrode-forming parts for use as electrodes that detect a current across the nano-gap when a target species is disposed therebetween.

54. (canceled)

55. The method of claim 53, wherein the compound-generating layer is a silicide-generating layer, wherein (c) comprises a silicidation of the electrode-forming parts during the reaction, and wherein the electrode-forming parts expand volumetrically during the silicidation.

56. (canceled)

57. (canceled)

58. (canceled)

59. (canceled)

60. The method of claim 53, wherein (c) comprises the reaction between the compound-generating layer and both of the electrode-forming parts.

61. The method of claim 53, wherein (c) comprises the reaction between the compound-generating layer and only one of the electrode-forming parts.

62. (canceled)

63. (canceled)

64. A nano-gap electrode sensor comprising at least two electrode parts disposed oppositely across a nano-gap on a substrate, wherein opposed sidewalls of the electrode parts gradually come closer to each other and a width between the sidewalls narrows gradually, and wherein the electrodes are adapted to detect a current across the nano-gap when a target species is disposed therebetween.

65. (canceled)

66. The nano-gap electrode sensor of claim 64, wherein the nano-gap is formed into a trailing curved shape in which the distance between the sidewalls of the electrode parts widens gradually as the nano-gap approaches the substrate.

67. The nano-gap electrode sensor of claim 64, wherein the sidewalls include outwardly expanding portions in contact with the substrate.

68. (canceled)

69. (canceled)
